(19) United States
(12) Patent Application Publication
(10) Pub. No.: US 2019/0144846 A1
(43) Pub. Date: May 16, 2019

(54) Harnessing heterologous and endogenous CRISPR-Cas machineries for efficient markerless genome editing in Clostridium

(71) Applicant: Neemo Inc, Hamilton (CA)

(72) Inventors: Michael E. Pyne, Montreal (CA); Mark Bruder, Elmira (CA); Murray Moo-Young, Waterloo (CA); Duane Chung, Oakville (CA); C. Perry Chou, Waterloo (CA)

(73) Assignee: Neemo Inc, Hamilton, ON (CA)

(21) Appl. No.: 16/098,035

(22) PCT Filed: Jul. 4, 2017
(86) PCT No.: PCT/CA2017/050805

§ 371 Date: Oct. 31, 2018

Related U.S. Application Data
(60) Provisional application No. 62/330,195, filed on May 1, 2016.

(51) Int. Cl. C12N 15/10; C12N 15/90

(52) U.S. Cl. CPC C12N 15/102; C12N 2310/20; C12N 2800/80; C12N 15/902

(57) ABSTRACT

By this invention, for the first time, a method for high-efficiency site-specific genetic engineering, utilizing either native or heterologous CRISPR-Cas9 systems, in the anaerobic bacterium Clostridium pasteurianum, is provided. Application of CRISPR-Cas9 systems has revolutionized genome editing across all domains of life. Here we report implementation of the heterologous Type CRISPR-Cas9 system in Clostridium pasteurianum for markerless genome editing. Since 74% of species harbor CRISPR-Cas loci in Clostridium, we also explored the prospect of co-opting host-encoded CRISPR-Cas machinery for genome editing. Motivation for this work was bolstered from the observation that plasmids expressing heterologous cas9 result in poor transformation of Clostridium. To address this barrier and establish proof-of-concept, we focus on characterization and exploitation of the C. pasteurianum Type CRISPR-Cas system. In silico spacer analysis and in vivo interference assays revealed three protospacer adjacent motif (PAM) sequences required for site-specific nucleolytic attack. Introduction of a synthetic CRISPR array and cpaAIR gene deletion template yielded an editing efficiency of 100%. In contrast, the heterologous Type II CRISPR-Cas9 system generated only 25% of the total yield of edited cells, suggesting that native machinery provides a superior foundation for genome editing by precluding expression of cas9 in trans. To broaden our approach, we also identified putative PAM sequences in three key species of Clostridium. This is the first report of genome editing through harnessing native CRISPR-Cas machinery in Clostridium.

This application claims the benefit of U.S. Provisional Patent Application No. 62/330,195, filed May. 1, 2016, which is incorporated by reference in its entirety.

### REFERENCES CITED

### Other References

Al-Hinai, M. A., Fast, A. G. & Papoutsakis, E. T. Novel system for efficient isolation of Clostridium double-crossover allelic exchange mutants enabling markerless chromosomal gene deletions and DNA integration. Appl. Environ. Microbiol. 78, 8112-8121 (2012).
Altschul, S. F., Gish, W., Miller, W., Myers, E. W. & Lipman, D. J. Basic local alignment search tool. J. Mol. Biol. 215, 403-410 (1990).
Barrangou, R. et al. CRISPR provides acquired resistance against viruses in prokaryotes. Science 315, 1709-1712 (2007).
Barrangou, R. CRISPR-Cas systems and RNA- guided interference. Wiley Interdisciplinary Reviews: RNA 4, 267-278 (2013).
Barrangou, R. & Marraffini, L. A. CRISPR-Cas systems: prokaryotes upgrade to adaptive immunity. Mol. Cell 54, 234-244 (2014).
Bhaya, D., Davison, M. & Barrangou, R. CRISPR-Cas systems in bacteria and archaea: versatile small RNAs for adaptive defense and regulation. Annu. Rev. Genet. 45, 273-297 (2011).
Bolotin, A., Quinquis, B., Sorokin, A. & Ehrlich, S. D. Clustered regularly interspaced short palindrome repeats (CRISPRs) have spacers of extrachromosomal origin. Microbiology 151, 2551-2561 (2005).
Boudry, P. et al. Function of the CRISPR-Cas system of the human pathogen Clostridium difficile. mBio 6, e01112-01115; doi:10.1128/mBio.01112-15 (2015).
Brouns, S. J. et al. Small CRISPR RNAs guide antiviral defense in prokaryotes. Science 321, 960-964 (2008).
Brown, S. D. et al. Comparison of single-molecule sequencing and hybrid approaches for finishing the genome of Clostridium autoethangenum and analysis of CRISPR systems in industrial relevant Clostridia. Biotechnol. Biofuels 7, 40; doi:10.1186/1754-6834-7-40 (2014).
Brüggemann, H. et al. Genomics of Clostridium tetani . Res. Microbiol. 166, 326-331 (2015).
Carte, J., Wang, R., Li, H., Terns, R. M. & Terns, M. P. Cas6 is an endoribonuclease that generates guide RNAs for invader defense in prokaryotes. Genes Dev. 22, 3489-3496 (2008).
Cartman, S. T., Kelly, M. L., Heeg, D., Heap, J. T. & Minton, N. P. Precise manipulation of the Clostridium difficile chromosome reveals a lack of association between the tcdC genotype and toxin production. Appl. Environ. Microbiol. 78, 4683-4690 (2012).
Charpentier, E. & Doudna, J. A. Biotechnology: Rewriting a genome. Nature 495, 50-51 (2013).

Cong, L. et al. Multiplex genome engineering using CRISPR/Cas systems. Science 339, 819-823 (2013).
Datsenko, K. A. & Wanner, B. L. One-step inactivation of chromosomal genes in Escherichia coli K-12 using PCR products. Proc. Natl. Acad. Sci. USA 97, 6640-6645 (2000).
Datta, S., Costantino, N., Zhou, X. M. & Court, D. L. Identification and analysis of recombineering functions from Gram-negative and Gram-positive bacteria and their phages. Proc. Natl. Acad. Sci. USA 105, 1626-1631 (2008).
Deltcheva, E. et al. CRISPR RNA maturation by trans-encoded small RNA and host factor RNase III. Nature 471, 602-607 (2011).
Deveau, H. et al. Phage response to CRISPR-encoded resistance in Streptococcus thermophilus. J. Bacteriol. 190, 1390-1400 (2008).
DiCarlo, J. E. et al. Genome engineering in Saccharomyces cerevisiae using CRISPR-Cas systems. Nucleic Acids Res. 41, 4336-4343 (2013).
Dong, H. J., Tao, W. W., Zhang, Y. P. & Li, Y. Development of an anhydrotetracycline-inducible gene expression system for solvent-producing Clostridium acetobutylicum: A useful tool for strain engineering. Metab. Eng. 14, 59-67 (2012).
Dong, H., Tao, W., Gong, F., Li, Y. & Zhang, Y. A functional recT gene for recombineering of Clostridium. J. Biotechnol. 173, 65-67 (2014).
Gasiunas, G., Barrangou, R., Horvath, P. & Siksnys, V. Cas9—crRNA ribonucleoprotein complex mediates specific DNA cleavage for adaptive immunity in bacteria. Proc. Natl. Acad. Sci. USA 109, E2579-E2586 (2012).
Godde, J. S. & Bickerton, A. The repetitive DNA elements called CRISPRs and their associated genes: evidence of horizontal transfer among prokaryotes. J. Mol. Evol. 62, 718-729 (2006).
Gomaa, A. A. et al. Programmable removal of bacterial strains by use of genome-targeting CRISPR-Cas systems. mBio 5, e00928-00913; doi:10.1128/mBio.00928-13 (2014).
Gratz, S. J. et al. Highly specific and efficient CRISPR/Cas9-catalyzed homology-directed repair in Drosophila. Genetics 196, 961-971 (2014).
Grissa, I., Vergnaud, G. & Pourcel, C. The CRISPRdb database and tools to display CRISPRs and to generate dictionaries of spacers and repeats. BMC Bioinformatics 8, 172; doi:10.1186/1471-2105-8-172 (2007).
Gudbergsdottir, S. et al. Dynamic properties of the Sulfolobus CRISPR/Cas and CRISPR/Cmr systems when challenged with vector-borne viral and plasmid genes and protospacers. Mol. Microbiol. 79, 35-49 (2011).
Haft, D. H., Selengut, J., Mongodin, E. F. & Nelson, K. E. A guild of 45 CRISPR-associated (Cas) protein families and multiple CRISPR/Cas subtypes exist in prokaryotic genomes. PLoS Comput Biol 1, e60; doi:doi:10.1371/journal.pcbi.0010060 (2005).
Hartman, A. H., Liu, H. L. & Melville, S. B. Construction and characterization of a lactose-inducible promoter system for controlled gene expression in Clostridium perfringens. Appl. Environ. Microbiol. 77, 471-478 (2011).
Hatheway, C. L. Toxigenic clostridia. Clin. Microbiol. Rev. 3, 66-98 (1990).
Heap, J. T., Pennington, O. J., Cartman, S. T. & Minton, N. P. A modular system for Clostridium shuttle plasmids. J. Microbiol. Methods 78, 79-85 (2009).
Heap, J. T. et al. The ClosTron: Mutagenesis in Clostridium refined and streamlined. J. Microbiol. Methods 80, 49-55 (2010).
Heap, J. T. et al. Integration of DNA into bacterial chromosomes from plasmids without a counter-selection marker. Nucleic Acids Res. 40, e59; doi:10.1093/nar/gkr1321 (2012).
Horwitz, A. A. et al. Efficient multiplexed integration of synergistic alleles and metabolic pathways in yeasts via CRISPR-Cas. Cell Systems 1, 88-96 (2015).
Hwang, W. Y. et al. Efficient genome editing in zebrafish using a CRISPR-Cas system. Nat. Biotechnol. 31, 227-229 (2013).
Jacobs, J. Z., Ciccaglione, K. M., Tournier, V. & Zaratiegui, M. Implementation of the CRISPR-Cas9 system in fission yeast. Nat. Commun. 5, 5344; doi:10.1038/ncomms6344 (2014).
Jiang, W., Brueggeman, A. J., Horken, K. M., Plucinak, T. M. & Weeks, D. P. Successful transient expression of Cas9 and single guide RNA genes in Chlamydomonas reinhardtii. Eukaryot. Cell 13, 1465-1469 (2014).
Jiang, W. Y., Bikard, D., Cox, D., Zhang, F. & Marraffini, L. A. RNA-guided editing of bacterial genomes using CRISPR-Cas systems. Nat. Biotechnol. 31, 233-239 (2013).
Jiang, Y. et al. Multigene editing in the Escherichia coli genome via the CRISPR-Cas9 system. Appl. Environ. Microbiol. 81, 2506-2514 (2015).
Jinek, M. et al. A programmable dual-RNA—guided DNA endonuclease in adaptive bacterial immunity. Science 337, 816-821 (2012).
Johnson, D. T. & Taconi, K. A. The glycerin glut: Options for the value-added conversion of crude glycerol resulting from biodiesel production. Environ. Prog. 26, 338-348 (2007).
Li, Y. et al. Harnessing Type I and Type III CRISPR-Cas systems for genome editing. Nucleic Acids Res. 44, e34; doi:10.1093/nar/gkv1044 (2015).
Li, Y. et al. Metabolic engineering of Escherichia coli using CRISPR—Cas9 meditated genome editing. Metab. Eng. 31, 13-21 (2015).
Luo, M. L., Leenay, R. T. & Beisel, C. L. Current and future prospects for CRISPR-based tools in bacteria. Biotechnol. Bioeng.; doi:10.1002/bit.25851 (2015).
Luo, M. L., Mullis, A. S., Leenay, R. T. & Beisel, C. L. Repurposing endogenous type I CRISPR-Cas systems for programmable gene repression. Nucleic Acids Res. 43, 674-681 (2015). Makarova, K. S. et al. Evolution and classification of the CRISPR—Cas systems. Nat. Rev. Microbiol. 9, 467-477 (2011).
Makarova, K. S. et al. An updated evolutionary classification of CRISPR-Cas systems. Nat. Rev. Microbiol. 13, 722-736 (2015).
Mali, P. et al. RNA-guided human genome engineering via Cas9. Science 339, 823-826 (2013).
Mojica, F. M., Diez-Villaseñor, C. s., Garcia-Martinez, J. & Soria, E. Intervening sequences of regularly spaced prokaryotic repeats derive from foreign genetic elements. J. Mol. Evol. 60, 174-182 (2005).

Mojica, F., Diez-Villasenor, C., Garcia-Martinez, J. & Almendros, C. Short motif sequences determine the targets of the prokaryotic CRISPR defence system. Microbiology 155, 733-740 (2009).
Nuñez, J. K. et al. Cas1-Cas2 complex formation mediates spacer acquisition during CRISPR—Cas adaptive immunity. Nat. Struct. Mol. Biol. 21, 528-534 (2014).
Olson, D. G. & Lynd, L. R. Transformation of Clostridium thermocellum by electroporation. Methods Enzymol. 510, 317-330 (2012).
Peng, D., Kurup, S. P., Yao, P. Y., Minning, T. A. & Tarleton, R. L. CRISPR-Cas9-mediated single-gene and gene family disruption in Trypanosoma cruzi. mBio 6, e02097-02014; doi:10.1128/mBio.02097-14 (2015).
Pourcel, C., Salvignol, G. & Vergnaud, G. CRISPR elements in Yersinia pestis acquire new repeats by preferential uptake of bacteriophage DNA, and provide additional tools for evolutionary studies. Microbiology 151, 653-663 (2005).
Pyne, M. E., Moo-Young, M., Chung, D. A. & Chou, C. P. Development of an electrotransformation protocol for genetic manipulation of Clostridium pasteurianum. Biotechnol. Biofuels 6, 50; doi:10.1186/1754-6834-6-50 (2013).
Pyne, M. E., Bruder, M., Moo-Young, M., Chung, D. A. & Chou, C. P. Technical guide for genetic advancement of underdeveloped and intractable Clostridium. Biotechnol. Adv. 32, 623-641 (2014).
Pyne, M. E., Moo-Young, M., Chung, D. A. & Chou, C. P. Expansion of the genetic toolkit for metabolic engineering of Clostridium pasteurianum : chromosomal gene disruption of the endogenous CpaAI restriction enzyme. Biotechnol. Biofuels 7, 163; doi:10.1186/s13068-014-0163-1 (2014).
Pyne, M. E. et al. Improved draft genome sequence of Clostridium pasteurianum strain ATCC 6013 (DSM 525) using a hybrid next-generation sequencing approach. Genome Announc. 2, e00790-00714; doi:10.1128/genomeA.00790-14 (2014).
Pyne, M. E., Moo-Young, M., Chung, D. A. & Chou, C. P. Coupling the CRISPR/Cas9 system with lambda Red recombineering enables simplified chromosomal gene replacement in Escherichia coli. Appl. Environ. Microbiol. 81, 5103-5114 (2015).
Sambrook, J., Fritsch, E. F. & Maniatis, T. Molecular cloning. Vol. 2 (Cold spring harbor laboratory press N.Y., 1989).
Sandoval, N. R., Venkataramanan, K. P., Groth, T. S. & Papoutsakis, E. T. Whole-genome sequence of an evolved Clostridium pasteurianum strain reveals Spo0A deficiency responsible for increased butanol production and superior growth. Biotechnol. Biofuels 8, 227; doi:10.1186/s13068-015-0408-7 (2015).
Sebo, Z. L., Lee, H. B., Peng, Y. & Guo, Y. A simplified and efficient germ line-specific CRISPR/Cas9 system for Drosophila genomic engineering. Fly 8, 52-57 (2014).
Semenova, E. et al. Interference by clustered regularly interspaced short palindromic repeat (CRISPR) RNA is governed by a seed sequence. Proc. Natl. Acad. Sci. USA 108, 10098-10103 (2011).
Shah, S. A., Erdmann, S., Mojica, F. J. & Garrett, R. A. Protospacer recognition motifs:

mixed identities and functional diversity. RNA biology 10, 891-899 (2013).
Shan, Q. et al. Targeted genome modification of crop plants using a CRISPR-Cas system. Nat. Biotechnol. 31, 686-688 (2013).
Shmakov, S. et al. Discovery and functional characterization of diverse class 2 CRISPR-Cas systems. Mol. Cell 60, 385-397 (2015).
Sinkunas, T. et al. Cas3 is a single-stranded DNA nuclease and ATP-dependent helicase in the CRISPR/Cas immune system. The EMBO journal 30, 1335-1342 (2011).
Sorek, R., Lawrence, C. M. & Wiedenheft, B. CRISPR-mediated adaptive immune systems in bacteria and archaea. Annu. Rev. Biochem. 82, 237-266 (2013).
Stoll, B. et al. Requirements for a successful defence reaction by the CRISPR-Cas subtype IB system. Biochem. Soc. Trans 41, 1444-1448 (2013).
Tracy, B. P., Jones, S. W., Fast, A. G., Indurthi, D. C. & Papoutsakis, E. T. Clostridia: The importance of their exceptional substrate and metabolite diversity for biofuel and biorefinery applications. Curr. Opin. Biotechnol. 23, 364-381 (2012). van der Oost, J., Jore, M. M., Westra, E. R., Lundgren, M. & Brouns, S. J. CRISPR-based adaptive and heritable immunity in prokaryotes. Trends Biochem. Sci. 34, 401-407 (2009).
Van Mellaert, L., Barbe, S. & Anne, J. Clostridium spores as anti-tumour agents. Trends Microbiol. 14, 190-196 (2006).
Vandewalle, K. Building genome-wide mutant resources in slow-growing mycobacteria, PhD thesis, Ghent University (2015).
Vercoe, R. B. et al. Cytotoxic chromosomal targeting by CRISPR/Cas systems can reshape bacterial genomes and expel or remodel pathogenicity islands. PLoS Genet 9, e1003454; doi:10.1371/journal.pgen.1003454 (2013).
Wang, H. et al. One-step generation of mice carrying mutations in multiple genes by CRISPR/Cas-mediated genome engineering. Cell 153, 910-918 (2013).

Wang, Y. et al. Markerless chromosomal gene deletion in Clostridium beijerinckii using CRISPR/Cas9 system. J. Biotechnol. 200, 1-5 (2015).
Westra, E. R. et al. CRISPR immunity relies on the consecutive binding and degradation of negatively supercoiled invader DNA by Cascade and Cas3. Mol. Cell 46, 595-605 (2012).
Xu, T. et al. Efficient genome editing in Clostridium cellulolyticum via CRISPR-Cas9 nickase. Appl. Environ. Microbiol. 81, 4423-4431 (2015).
Yazdani, S. S. & Gonzalez, R. Anaerobic fermentation of glycerol: A path to economic viability for the biofuels industry. Curr. Opin. Biotechnol. 18, 213-219 (2007).
Zebec, Z., Manica, A., Zhang, J., White, M. F. & Schleper, C. CRISPR-mediated targeted mRNA degradation in the archaeon Sulfolobus solfataricus. Nucleic Acids Res. 42, 5280-5288 (2014).
Zhou, Y., Liang, Y., Lynch, K. H., Dennis, J. J. & Wishart, D. S. PHAST: A fast phage search tool. Nucleic Acids Res. 39, W347-W352; doi:10.1093/nar/gkr485 (2011).

### TECHNICAL FIELD

The present invention is directed to bacterial cells and methods for making genetic modifications within bacterial cells, and methods and nucleic acids related thereto.

### BACKGROUND

Clustered Regularly Interspaced Short Palindromic Repeats (CRISPRs) and CRISPR-associated (Cas) proteins comprise the basis of adaptive immunity in bacteria and archaea (Barrangou, 2014; Sorek, et al, 2013). CRISPR-Cas systems are currently grouped into six broad types, designated Type I through VI (Makarova, et al, 2015; Shmakov, et al, 2015). CRISPR-Cas Types I, II, and III, the most prevalent systems in both archaea and bacteria (Makarova, et al, 2015), are differentiated by the presence of cas3, cas9, or cas10 signature genes, respectively (Makarova, et al, 2011). Based on the composition and arrangement of cas gene operons, CRISPR-Cas systems are further divided into 16 distinct subtypes (Makarova, et al, 2015). Type I systems, comprised of six distinct subtypes (I-A to I-F), exhibit the greatest diversity (Haft, et al, 2005) and subtype I-B is the most abundant CRISPR-Cas system represented in nature (Makarova, et al, 2015). CRISPR-Cas loci have been identified in 45% of bacteria and 84% of archaea (Grissa, et al, 2007) due to widespread horizontal transfer of CRISPR-Cas loci within the prokaryotes (Godde, 2006).

CRISPR-based immunity encompasses three distinct processes, termed adaptation, expression, and interference (Barrangou, 2013; van der Oost, et al, 2009). Adaptation involves the acquisition of specific nucleotide sequence tags, referred to as protospacers in their native context within invading genetic elements, particularly bacteriophages (phages) and plasm ids (Bolotin, et al, 2005; Mojica, et al, 2005; Pourcel, et al, 2005). During periods of predation, protospacers are rapidly acquired and incorporated into the host genome, where they are subsequently referred to as spacers (Barrangou, et al, 2007). Cas1 and Cas2, which form a complex that mediates acquisition of new spacers (Nuñez, et al, 2014), are the only proteins conserved between all CRISPR-Cas subtypes (Makarova, et al, 2011). Chromosomally-encoded spacers are flanked by 24-48 bp partially-palindromic direct repeat sequences (Haft, et al, 2005), iterations of which constitute CRISPR arrays. Up to 587 spacers have been identified within a single CRISPR array (Bhaya, et al, 2011), exemplifying the exceptional level of attack experienced by many microorganisms in nature. During the expression phase of CRISPR immunity, acquired spacer sequences are expressed and, in conjunction with Cas proteins, provide resistance against invading genetic elements. CRISPR arrays are first transcribed into a single precursor CRISPR RNA (pre-crRNA), which is cleaved into individual repeat-spacer-repeat units by Cas6 (Type I and III systems) (Carte, et al, 2008) or the ubiquitous RNase III enzyme and a small trans-activating crRNA (tracrRNA) (Type II systems) (Deltcheva, et al, 2011), yielding mature crRNAs (FIG. 1). Once processed, crRNAs enlist and form complexes with specific Cas proteins, including the endonucleases responsible for attack of invading nucleic acids during the interference stage of CRISPR immunity. In Type I systems, crRNAs complex with ‘Cascade’ (a multiprotein Cas complex for antiviral defence) and base pair with invader DNA (Brouns, et al, 2008), triggering nucleolytic attack by Cas3 (Sinkunas, et al, 2011). In many CRISPR-Cas subtypes, Cascade includes Cas5, Cas6, Cas7, and Cas8 (Haft, et al, 2005). Type II systems are markedly simpler and more compact than Type I machinery, as the Cas9 endonuclease, tracrRNA, and crRNA, as well as the ubiquitous RNase III enzyme, are the sole determinants required for interference (FIG. 1). Alternatively, crRNA and tracrRNAs can be fused into a single guide RNA (gRNA) (Jinek, et al, 2012). While Cas9 attack results in a blunt double-stranded DNA break (DB) (Gasiunas, et al, 2012), Cas3 cleaves only one strand of invading DNA, generating a DNA nick (DN). Nicked target DNA is subsequently unwound and progressively degraded by Cas3 (Westra, et al, 2012). Because host-encoded spacer and invader protospacer sequences are often identical, cells harboring Type I and II CRISPR-Cas systems evade self-attack through recognition of a requisite sequence located directly adjacent to invading protospacers, termed the protospacer-adjacent motif (PAM) (Deveau, et al, 2008; Mojica, et al, 2009). In many organisms, the PAM element is highly promiscuous, affording flexibility in recognition of invading protospacers, whereby specific non-degenerate sequences that constitute the consensus are referred to as PAM sequences. The location of the PAM differs between Type I and II CRISPR-Cas systems, occurring immediately upstream of the protospacer in Type I (i.e. 5′-PAM-protospacer-3′) and immediately downstream of the protospacer in Type II systems (i.e. 5′-protospacer-PAM-3′) (Barrangou, et al, 2007; Mojica, et al, 2009; Shah, et al, 2013) (FIG. 1). The site of nucleolytic attack also differs between CRISPR-Cas Types, as Cas9 cleaves DNA three nucleotides upstream of the PAM element (Jinek, et al, 2012; Gasiunas, et al, 2012), while Cas3 nicks the PAM-complementary strand outside of the area of interaction with crRNA (Sinkunas, et al, 2011).

Owing to the simplicity of CRISPR-Cas9 interference in Type II systems, the S. pyogenes CRISPR-Cas9 machinery has recently been implemented for extensive genome editing in a wide range of organisms, such as E. coli (Jiang, et al, 2013; Jiang, et al, 2015; Pyne, et al, 2015), yeast (DiCarlo, et al, 2013; Horwitz, et al, 2015), mice (Wang, et al, 2013), zebrafish (Hwang, et al, 2013), plants (Shan, et al, 2013), and human cells (Cong, et al, 2013; Mali, et al, 2013). In bacteria, CRISPR-based methods of genome editing signify a critical divergence from traditional techniques of genetic manipulation involving the use of chromosomally-encoded antibiotic resistance markers, which must be excised and recycled following each successive round of integration (Datsenko, 2000). Within Clostridium, a genus with immense importance to medical and industrial biotechnology (Tracy, et al, 2012; Van Mellaert, et al, 2006), as well as human disease (Hatheway, 1990), genetic engineering technologies are notoriously immature, as the genus suffers from overall low transformation efficiencies and poor homologous recombination (Pyne, Bruder, et al, 2014). Existing clostridial genome engineering methods, based on mobile group II introns, antibiotic resistance determinants, and counter-selectable markers, are laborious, technically challenging, and often ineffective (Al-Hinai, et al, 2012; Heap, et al, 2012; Heap, et al, 2010). In contrast, CRISPR-based methodologies provide a powerful means of selecting rare recombination events, even in strains suffering from poor homologous recombination. Such strategies have been shown to be highly robust, frequently generating editing efficiencies up to 100% (Jiang, et al, 2013; Pyne, et al, 2015; Li, et al, Metab. Eng., 2015). Accordingly, the S. pyogenes Type II CRISPR-Cas system has recently been adapted for use in C. beijerinckii (Wang, et al, 2015) and C. cellulolyticum (Xu, et al, 2015), facilitating highly precise genetic modification of clostridial genomes and paving the way for robust genome editing in industrial and pathogenic clostridia.

Here we report development of broadly applicable strategies of markerless genome editing based on exploitation of both heterologous (Type II) and endogenous (Type I) bacterial CRISPR-Cas systems in C. pasteurianum, an organism possessing substantial biotechnological potential for conversion of waste glycerol to butanol as a prospective biofuel (Johnson, 2007). While various tools for genetic manipulation of C. pasteurianum are under active development recently (Pyne, et al, 2013; Pyne, Moo-Young, et al, 2014), effective site-specific genome editing for this organism is lacking. In this study, we demonstrate the first implementation of S. pyogenes Type II CRISPR-Cas9 machinery for markerless and site-specific genome editing in C. pasteurianum. Recently, we sequenced the C. pasteurianum genome (Pyne, et al, Genome Announc., 2014) and identified a central Type I-B CRISPR-Cas locus, which we exploit here as a chassis for genome editing based on earlier successes harnessing endogenous CRISPR-Cas loci in other bacteria (Li, et al, Nucleic Acids Res, 2015; Luo, Leenay, 2015). Our strategy encompasses plasm id-borne expression of a synthetic Type I-B CRISPR array that can be site-specifically programmed to any gene within the organism's genome. Providing an editing template designed to delete the chromosomal protospacer and adjacent PAM yields an editing efficiency of 100% based on screening of 10 representative colonies. To our knowledge, the approach described here is the first report of genome editing in Clostridium by co-opting native CRISPR-Cas machinery. Importantly, our strategy is broadly applicable to any bacterium or archaeon that encodes a functional CRISPR-Cas locus and appears to yield more edited cells compared to the commonly employed heterologous Type II CRISPR-Cas9 system.

### SUMMARY OF THE INVENTION

The present invention provides protocols that enable manipulation of the genome of bacterial cells.

In one preferred embodiment, the protocols for genome manipulation involve the use of heterologous or endogenous Clustered Regularly Interspaced Short Palindromic Repeat (CRISPR) tools. In a further preferred embodiment, the genome manipulations include, but are limited to, insertions of DNA into the bacterial genome, deletions of DNA from the bacterial genome, and the introduction of mutations within the bacterial genome. The term ‘genome’ encompasses both native and modified chromosomal and episomal genetic units, as well as non-native, introduced genetic units.

In a preferred embodiment, the bacterial cells are from the genus Clostridium. In a further preferred embodiment, the bacterial cells are from the bacterium Clostridium pasteurianum. In another preferred embodiment, the bacterial cells are selected from the group consisting of Clostridium autoethanogenum, Clostridium tetani, and Clostridium thermocellum.

In a preferred embodiment, the heterologous CRISPR system involves the use of the Stretococcus pyogenes cas9 enzyme.

In a preferred embodiment, the endogenous CRISPR system involves the use of the native CRISPR system within the bacterium Clostridium pasteurianum.

In one preferred embodiment, the use of the endogenous CRISPR system of Clostridium pasteurianum involves the use of direct repeat sequences selected from the group consisting of SEQ ID NO. 43 and SEQ ID NO 45, and a 5′ protospacer adjacent motif (PAM) selected from the group consisting of 5′-TTTCA-3′, 5′-AATTG-3′, 5′-TATCT-3′. In another preferred embodiment, the 5′ PAM sequence is selected from the group consisting of 5′-AATTA-3′, 5′-AATTT-3′, 5′-TTTCT-3′, 5′-TCTCA-3′, 5′-TCTCG-3′, and 5′-TTTCA-3′. In another preferred embodiment, the 5′ PAM sequence is selected from the group consisting of 5′-TCA-3′, 5′-TTG-3′, and 5′-TCT-3′.

In one preferred embodiment, where the bacterial cell is selected from the group consisting of Clostridium autoethanogenum, Clostridium tetani, and Clostridium thermocellum, the direct repeats utilized in the invention are taken from the native CRISPR arrays of each bacterial cell, in particular, the direct repeats are taken from SEQ ID NO 46 and SEQ ID NO 47 when the bacterial cell is Clostridium autoethanogenum, from SEQ ID NO 48, SEQ ID NO 49, and SEQ ID NO 50 when the bacterial cell is Clostridium tetani, and from SEQ ID NO 51, SEQ ID NO 52, and SEQ ID NO 53 when the bacterial cell is Clostridium thermocellum.

In another preferred embodiment, when the bacterial cell is Clostridium autoethanogenum, the 5′ PAM sequence is selected from the group consisting of 5′-ATTAA-3′, 5′-ACTAA-3′, 5′-AAGAA-3′, 5′-ATCAA-3′, and 5′-NAA-3′, where ‘N’ can be any of ‘A’, ‘C’, ‘G’, and ‘T’ nucleotides.

In another preferred embodiment, when the bacterial cell is Clostridium tetani, the 5′ PAM sequence is selected from the group consisting of 5′-TTTTA-3′, 5′-TATAA-3′, 5′-CATCA-3′, and 5′-TNA-3′, where ‘N’ can be any of ‘A’, ‘C’, ‘G’, and ‘T’ nucleotides.

In another preferred embodiment, when the bacterial cell is Clostridium thermocellum, the 5′ PAM sequence is selected from the group consisting of 5′-TTTCA-3′, 5′-GGACA-3′, 5′-AATCA-3′, and 5′-NCA-3′, where ‘N’ can be any of ‘A’, ‘C’, ‘G’, and ‘T’ nucleotides.

The present invention also includes bacterial cells containing genomes that have been modified using one of the above mentioned protocols involving CRISPR tools. The present invention also includes a protocol for rapidly determining a candidate pool of PAM sequences for any bacteria that includes one or more components of a native CRISPR system, wherein said pool of candidate PAM sequences may be directly assayed for their ability to enable the utilization of the native CRISPR system, thereby avoiding the labour intensity of an exhaustive, empirical search through plasmid or oligonucleotide libraries representing the space of potential PAM sequences.

### DESCRIPTION OF THE FIGURES

FIG. 1 Comparison of Type I (left) and Type II (right) CRISPR-Cas interference mechanisms. CRISPR arrays, comprised of direct repeats (DRs; royal blue and dark green) and spacer tags (light blue and light green) are first transcribed into a single large pre-crRNA by a promoter located within the CRISPR leader (lead). The resulting transcript is cleaved and processed into individual mature crRNAs by the Cas6 endonuclease (Type I systems) or the ubiquitous RNase III enzyme (Type II systems). Processing is mediated by characteristic secondary structures (hairpins) formed by Type I pre-crRNAs or by a trans-activating RNA (tracrRNA; brown) possessing homology to direct repeat sequences in Type II systems. A single synthetic guide RNA (gRNA) can replace the dual crRNA-tracrRNA interaction (not shown). Mature crRNAs are guided to invading nucleic acids through homology between crRNAs and the corresponding invader protospacer sequence. Type I interference requires the multiprotein Cascade complex (comprised of cas6-cas8b-cas7-cas5 in Clostridium difficile (Boudry, et al, 2015) and C. pasteurianum), encoded downstream of the Type I CRISPR array. Type I and II interference mechanisms require recognition of one of multiple protospacer adjacent motif (PAM) sequences, which collectively comprise the consensus PAM element (red). The location of the PAM and the site of nucleolytic attack relative to the protospacer sequence differs between Type I and II CRISPR-Cas systems. Representative PAM sequences from C. difficile (Type I-B) (Boudry, et al, 2015) and Streptococcus pyogenes (Type II) (Mojica, et al, 2009) CRISPR-Cas loci are shown. Nucleolytic attack by Cas3 or Cas9 results in a DNA nick (DN) or blunt double-stranded DNA break (DB), respectively. Both CRISPR-Cas loci contain cas4 and cas2 genes (not shown), while the Type I and II loci also contain cas4 and csn2 genes, respectively (not shown).

FIG. 2 Genome editing in C. pasteurianum using the heterologous S. pyogenes Type II CRISPR-Cas9 system. (a) cpaAIR gene deletion strategy using Type II CRISPR-Cas9. Introduction of a double-stranded DB to the cpaAIR locus was achieved by programming a gRNA spacer sequence (green) and expressing heterologous cas9 within plasmid pCas9gRNA-cpaAIR. cpaAIR-targeted gRNA, containing cas9 binding handle (orange), is directed to the chromosomal cpaAIR gene through base-pairing to the protospacer sequence and Cas9-recognition of the S. pyogenes PAM element (5′-NGG-3′; red). Insertion of a cpaAIR gene editing cassette in pCas9gRNA-cpaAIR, generating pCas9gRNA-delcpaAIR, leads to homologous recombination and deletion of a portion of the cpaAIR coding sequence, including the protospacer and PAM elements. Unmodified cells are selected against by Cas9 cleavage, while edited cells possessing a partial cpaAIR deletion are able evade attack. Genes, genomic regions, and plasm ids are not depicted to scale. (b) Transformation efficiency corresponding to Type II CRISPR-Cas9 vectors (pCas9gRNA-cpaAIR and pCas9gRNA-delcpaAIR) and various cas9 expression derivatives and control constructs (pMTL85141, p85Cas9, p83Cas9, p85delCas9). Transformation efficiency is reported as the number of CFU generated per μg of plasmid DNA. Data shown are averages resulting from at least two independent experiments and error bars depict standard deviation. (c) Colony PCR genotyping of pCas9gRNA-delcpaAIR transformants. Primers cpaAIR.S and cpaAIR.AS were utilized in colony PCR to screen 10 colonies harboring pCas9gRNA-delcpaAIR. Expected product sizes are shown corresponding to the wild-type (2,913 bp) and the cpaAIR deletion mutant (2,151 bp) strains of C. pasteurianum. Lane 1: linear DNA marker; lane 2: no colony control; lanes 3: wild-type colony; 4: colony harboring pCas9gRNA-cpaAIR; lanes 5-14: colonies harboring pCas9gRNA-delcpaAIR.

FIG. 3 Characterization of the central Type I-B CRISPR-Cas system of C. pasteurianum. (a) Genomic structure of the Type I-B CRISPR-Cas locus of C. pasteurianum. The central CRISPR-Cas locus is comprised of 37 distinct spacers (light blue) flanked by 30 nt direct repeats (royal blue) and a representative Type I-B cas operon containing cash-cas8b-cas7-cas5-cas3-cas4-cas1-cas2 (abbreviated cas68b753412). A promoter within the putative leader sequence (lead) drives transcription of the CRISPR array. (b) Plasmid interference assays using protospacers 18, 24, and 30 (uppercase) and different combinations of 5′ and/or 3′ protospacer-adjacent sequence (lowercase). Protospacers were designed to possess no adjacent sequences, 5′ or 3′ adjacent sequence, or both 5′ and 3′ adjacent sequences. Protospacers were cloned in plasmid pMTL85141 and the resulting plasm ids were used to transform C. pasteurianum. Putative PAM sequences are underlined. Pictures of representative transformants are shown corresponding to protospacer 30.

FIG. 4 Genome editing in C. pasteurianum using the endogenous Type I-B CRISPR-Cas system. (a) cpaAIR gene deletion strategy using endogenous Type I-B CRISPR-Cas machinery. A condensed C. pasteurianum Type I-B CRISPR array (array) and cas gene operon (cas) is shown, in addition to the cpaAIR targeting locus. An inset is provided showing the full-length C. pasteurianum CRISPR-Cas locus comprised of a 37-spacer array and cas operon containing cas6-cas8b-cas7-cas5-cas3-cas4-cas1-cas2 (abbreviated cas68b753412). Introduction of a DNA nick to the cpaAIR gene was achieved by expressing a synthetic CRISPR array containing a 36 nt cpaAIR spacer (green) flanked by 30 nt direct repeats (royal blue) within plasmid pCParray-cpaAIR. The synthetic array is transcribed into pre-crRNA and processed into mature crRNA by Casio. crRNA processing and interference occurs as depicted in FIG. 1. In some experiments, selection against wild-type cells using pCParray-cpaAIR generated a single background colony. Insertion of a cpaAIR gene editing cassette in pCParray-cpaAIR, generating pCParray-delcpaAIR, leads to homologous recombination and deletion of a portion of the cpaAIR coding sequence, including the protospacer and PAM sequence (5′-AATTG-3′). Unmodified cells are selected against by Cas3 cleavage, while edited cells possessing a partial cpaAIR deletion are able to survive. Genes, genomic regions, and plasm ids are not depicted to scale. (b) Transformation efficiency corresponding to Type I-B CRISPR-Cas vectors. Transformation efficiency is reported as the number of CFU generated per pg of plasmid DNA. Data shown are averages resulting from at least two independent experiments and error bars depict standard deviation. (c) Colony PCR genotyping of pCParray-delcpaAIR transformants. Primers cpaAIR.S and cpaAIR.AS were utilized in colony PCR to screen 10 colonies harboring pCParray-delcpaAIR. Expected product sizes are shown corresponding to the wild-type (2,913 bp) and the cpaAIR deletion mutant (2,151 bp) strains of C. pasteurianum. Lane 1: linear DNA marker; lane 2: no colony control; lanes 3: wild-type colony; 4: colony harboring pCParray-cpaAIR; lanes 5-14: colonies harboring pCParray-delcpaAIR.

FIG. 5. Sequence and structure of synthetic DNA constructs employed in this study. (a) 821 bp synthetic gRNA gene synthesis product targeted to the C. pasteurianum cpaAIR locus. The synthetic gRNA containing a 20 nt cpaAIR spacer tag (green) and cas9 binding handle (orange) was expressed from the sCbei_5830 small RNA promoter (P_{sCbei}_{_}₅₈₃₀). A reverse orientation C. pasteurianum thl gene promoter (Pₜₕₗ) and partial cas9 coding sequence (violet) was included for transcriptional fusion of Pₜₕₗ to the cas9 gene. Promoter-containing regions are shown in uppercase letters and restriction endonuclease recognition sites utilized for cloning (SacII+BstZ17I) are underlined. (b) 667 bp synthetic CRISPR array gene synthesis product targeted to the C. pasteurianum cpaAIR locus. The synthetic CRISPR array containing a 37 nt cpaAIR spacer (green) flanked by 30 nt direct repeats (blue) was expressed from a putative promoter (not identified) within the CRISPR leader sequence (lead; red). Sacl recognition sites utilized for cloning are underlined.

TABLE 1
Putative protospacer matches identified through in silico analysis of C. pasteurianum CRISPR spacers

| Spacer | | | Mis- | Putative PAM |
|---|---|---|---|---|
| number | Spacer-protospacer match^{a} | Invading element^{b} | matches | sequence^{c} |
| 18 | GTAAAATTTGATTGTCCTCATTGCGATGAAGAAA | Clostridium pasteurianum | 4 | 5′-TTTCA-3′ |
| | ATAAAATTTGATTGCCCTCACTGTGATGAAGAAA | BC1 (vicinity of phage | | |
| | | genes) | | |
| 24 | TTGCAATAGAATGTGATAAAGACCATACTCATATGT | Clostridium phage | 2 | 5′-AATTG-3′ |
| | TTGCAATAGAATGCGATAAAGACCATACACATATGT | φCD211 | | |
| | TTGCAATAGAATGTGATAAAGACCATACTCATATGT | Clostridium acidurici 9a | 4 | 5′-AATTA-3′ |
| | TAGCAATAGAATGTGATAGAGATCATACGCATATGT | (transposase) | | |
| | TTGCAATAGAATGTGATAAAGACCATACTCATATGT | Clostridium aceticum | 7 | 5′-AATTT-3′ |
| | TGGCAATAGAATGTGATAAAGACCACTGCCATCTTT | strain DSM 1496 plasmid | | |
| | | CACET_5p (transposase) | | |
| 30 | ATAATATGGATTGAAGAGTGTTCAGAAGTTAAATA | Clostridium botulinum | 3 | 5′-TATCT-3′ |
| | ATAATATGGATAGAAGAATGTTCAGAAGTAAAATA | CDC_297 (intact | | |
| | | prophage) | | |
| | ATAATATGGATTGAAGAGTGTTCAGAAGTTAAATA | Clostridium pasteurianum | 3 | 5′-TTTCT-3′ |
| | TTAATATGGATAGAAGAATGTTCAGAAGTTAAATA | NRRL B-598 (intact | | |
| | | prophage) | | |
| | ATAATATGGATTGAAGAGTGTTCAGAAGTTAAATA | Bacillus licheniformis | 4 | 5′-TCTCA-3′ |
| | ATAATATGGATTGAGGAATGTTCAGAGGTCAAATA | ATCC 14580 (phage | | |
| | | terminase) | | |
| | ATAATATGGATTGAAGAGTGTTCAGAAGTTAAATA | Bacillus pumilus strain | 4 | 5′-TCTCG-3′ |
| | ATCATATGGATTGAGGAATGTTCAGAAGTTAAGTA | NJ-V2 (phage terminase) | | |
| | ATAATATGGATTGAAGAGTGTTCAGAAGTTAAATA | Bacillus subtilis strain | 5 | 5′-TTTCA-3′ |
| | TTAATATGGATTGAAGAGTGCTCAGAGGTGAAGTA | SG6 (intact prophage) | | |

^{a}Spacer-protospacer mismatches are underlined.
^{b}For hits found within bacterial genomes, the location of the protospacer sequence relative to prophage regions and mobile genetic elements is provided in parentheses.
^{c}5 nt of adjacent sequence is provided. PAM sequences corresponding to the top protospacer hit from each spacer (bolded) were selected for in vivo interference assays.

TABLE 2
Putative protospacer matches identified through in silico analysis of clostridial CRISPR spacers

| Organism | | | | |
|---|---|---|---|---|
| (CRISPR-Cas | | | Mis- | Putative PAM |
| subtype) | Spacer-protospacer match^{a} | Invading element^{b} | matches | sequence^{c} |
| C. autoethanogenum | AAGAGTTGATACTTTACTTATAGATTACTTAGGTGC | Clostridium | 0 | 5′-ATTAA-3′ |
| DSM 10061 (Type | AAGAGTTGATACTTTACTTATAGATTACTTAGGTGC | ljungdahlii DSM | | |
| I-B) | | 13528 (incomplete | | |
| | | prophage) | | |
| | TAGACCACAATTAAATGCAATGTTAGAATTTGCTCG | Clostridium phage | 4 | 5′-ACTAA-3′ |
| | TAGGCCACAATTAAAAGCCATGTTAGAATTTGCTAG | vB_CpeS-CP51 | | |
| | AAATACATTTTATAAATTATTAAAAGAATATGAGG | Bacillus | 4 | 5′-AAGAA-3′ |
| | AAATACTTTTTATAAAATATTGAAAGAATATGAAG | thuringiensis HD- | | |
| | | 789 plasmid | | |
| | | pBTHD789-3 | | |
| | GCAGCTCCAGGAGCAAAAACCAAAGGTACTATTCGC | Enterococcus | 8 | 5′-ATCAA-3′ |
| | GAAGCTCCAGGAGCAAAAATCAAAGGTATTTATTTT | durans strain | | |
| | | KLDS 6.0930 | | |
| | | (vicinity of | | |
| | | transposase and | | |
| | | phage genes) | | |
| C. tetani 12124569 | ATATTTCTTTTTTACTCCAATAAGCTCCAATGAG | Clostridium | 3 | 5′-TTTTA-3′ |
| (Type I-B) | ATATTTCTTTTTTACTCCAATCAGCCCCAATAAG | botulinum A2 str. | | |
| | | Kyoto (intact | | |
| | | prophage) | | |
| | AAAAGCCAATCAAAATCTATTTTATATTTAGATTT | Clostridium | 3 | 5′-TATAA-3′ |
| | AAAAGCCAGTCAAAATCTATTAAATATTTAGATTT | botulinum F str. | | |
| | | 230613 (intact | | |
| | | prophage) | | |
| | AAAGATAAGAGAGAAGGATTACTTCCAGAAGTAGC | Bacillus sp. HAT- | 7 | 5′-CATCA-3′ |
| | AAAGACAAGCGAGAAGGGTTGCTTCCAGAAGTCTA | 4402 (questionable | | |
| | | prophage) | | |
| C. thermocellum | ATTCGTTTATCTTTATCAAATCACTCCCTCCCTTCAG | Clostridium | 2 | 5′-TTTCA-3′ |
| ATCC 27405 (Type | ATTCGTTTGTCTTTATCAAATCACTCCCTCCTTTCAG | stercorarium | | |
| I-B) | | subsp. | | |
| | | stercorarium DSM | | |
| | | 8532 (intact | | |
| | | prophage) | | |
| | TGATGAAGGACGCTGAAACAGGAATGTTCCAGGCTG | Clostridium | 2 | 5′-GGACA-3′ |
| | TGATGAAGGACGCTGAAACAGGAATGTTTCAGGCCG | clariflavum DSM | | |
| | | 19732 (vicinity of | | |
| | | transposase) | | |
| | ACGAAGCAGGTTTATACAGTTTGATATTGAAATCAA | Staphylococcus | 6 | 5′-AATCA-3′ |
| | ACGAATCAGGTTTATACAGTTTAATCTTTTCATCAA | phage | | |
| | | vB_SauM_Remus | | |

^{a}Spacer-protospacer mismatches are underlined. In instances where multiple protospacer hits were obtained from a single spacer query, the top hit is provided. Generally, PAM sequences were found to be identical between multiple protospacer hits from a single spacer sequence.
^{b}For hits found within bacterial genomes, the location of the protospacer sequence relative to prophage regions and mobile genetic elements is provided in parentheses.
^{c}5 nt of adjacent sequence is provided. Potential conserved residues are bolded.

TABLE 3
Summary of clostridial Type I-B CRISPR-Cas loci analyzed to date

| | Number of | | | |
|---|---|---|---|---|
| | spacers | PAM | | |
| Species | (total)^{a} | sequences^{b} | PAM^{b} | Reference |
| C. autoethanogenum | 22, 43, 33 | 5′-TAA-3′ | 5′-NAA-3′ | This study; |
| DSM 10061 | (98) | 5′-TAA-3′ | | (Grissa, et al, |
| | | 5′-CAA-3′ | | 2007) |
| | | 5′-GAA-3′ | | |
| C. difficile | 1, 2, 1, 1, 4, | 5′-CCA-3′ | 5′-CCW-3′^{c} | (Boudry, et al, |
| 630/R20291 | 2, 4, 3, 2, 14, | 5′-CCT-3′ | | 2015; Grissa, et al, |
| | 11, 4, 5, 4, | | | 2007) |
| | 14, 9, 26, 9 | | | |
| | (116) | | | |
| C. pasteurianum | 37, 8 (45) | 5′-TCA-3′ | ND^{d} | This study; |
| ATCC 6013 | | 5′-TTG-3′ | | (Grissa, et al, |
| | | 5′-TCT-3′ | | 2007) |
| C. tetani 12124569 | 22, 3, 4, 2, 4, | 5′-TAA-3′ | 5′-TNA-3′ | This study; |
| | 5, 10, 3 (53) | 5′-TTA-3′ | | (Grissa, et al, |
| | | 5′-TCA-3′ | | 2007) |
| C. thermocellum | 51, 96, 169, | 5′-TCA-3′ | 5′-NCA-3′ | This study; |
| ATCC 27405 | 78, 42 (436) | 5′-TCA-3′ | | (Grissa, et al, |
| | | 5′-ACA-3′ | | 2007) |

^{a}Spacers corresponding to Type I-B CRISPR-Cas loci analyzed in this study are bolded.
^{b}3 nt PAM and PAM sequences are shown. Experimentally-verified motifs are bolded.
^{c}W = weak (A or T).
^{d}ND = not determined due to highly varied PAM sequences.

TABLE 4
Strains and plasmids employed in this study

| | Relevant characteristics | Source or reference |
|---|---|---|
| Strain | | |
| Escherichia coli DH5α | F⁻ endA1 glnV44 thi-1 recA1 relA1 gyrA96 deoR mpG | Lab stock |
| | φ80dlacZΔM15 Δ(lacZYA-argF)U169, hsdR17(rK⁻mK⁺), λ⁻ | |
| Escherichia coli ER1821 | F⁻ endA1 glnV44 thi-1 relA1? e14⁻(mcrA⁻) rfbD1? spoTT? | Lab stock; New England Biolabs |
| | Δ(mcrC-mrr)114::IS10 | |
| Clostridium pasteurianum | Wild-type | American Type Culture |
| ATCC 6013 | | Collection |
| Clostridium pasteurianum | Markerless cpaAIR deletion mutant | This study |
| ΔcpaAIR | | |
| Plasmid | | |
| pFnuDIIMKn | M.FnuDII methyltransferase plasmid for methylation of E. coli- | (Pyne, et al, 2013) |
| | C. pasteurianum shuttle vectors (Km^{R}; p15A ori) | |
| pMTL83151 | E. coli-Clostridium shuttle vector (Cm^{R}; ColE1 ori; pCB102 | (Heap, et al, 2009) |
| | ori) | |
| pMTL85141 | E. coli-Clostridium shuttle vector (Cm^{R}; ColE1 ori; pIM13 ori) | (Heap, et al, 2009) |
| pCas9 | E. coli cas9 and tracrRNA expression vector (Cm^{R}; p15A ori) | (Jiang, et al, 2013) |
| pCas9gRNA-cpaAIR | Type II CRISPR expression vector containing cas9 and gRNA | This study |
| | targeted to the C. pasteurianum cpaAIR gene | |
| pCas9gRNA-delcpaAIR | Type II CRISPR genome editing vector derived by inserting a | This study |
| | cpaAIR deletion editing cassette into pCas9gRNA-cpaAIR | |
| p85Cas9 | cas9 expression vector derived by inserting cas9 with its native | This study |
| | promoter from pCas9 into pMTL85141 | |
| p83Cas9 | cas9 expression vector derived by inserting cas9 and the | This study |
| | tracrRNA from pCas9 into pMTL83151 | |
| p85delCas9 | Derived by deleting the cas9 promoter from p85cas9 | This study |
| pSpacer18 | C. pasteurianum protospacer 18 construct lacking flanking | This study |
| | sequences | |
| pSpacer18-5′ | C. pasteurianum protospacer 18 construct including 5′ | This study |
| | protospacer-adjacent sequence | |
| pSpacer18-3′ | C. pasteurianum protospacer 18 construct including 3′ | This study |
| | protospacer-adjacent sequence | |
| pSpacer18-flank | C. pasteurianum protospacer 18 construct including flanking | This study |
| | protospacer-adjacent sequence | |
| pSpacer24 | C. pasteurianum protospacer 24 construct lacking flanking | This study |
| | sequences | |
| pSpacer24-5′ | C. pasteurianum protospacer 24 construct including 5′ | This study |
| | protospacer-adjacent sequence | |
| pSpacer24-3′ | C. pasteurianum protospacer 24 construct including 3′ | This study |
| | protospacer-adjacent sequence | |
| pSpacer24-flank | C. pasteurianum protospacer 24 construct including flanking | This study |
| | protospacer-adjacent sequence | |
| pSpacer30 | C. pasteurianum protospacer 30 construct lacking flanking | This study |
| | sequences | |
| pSpacer30-5′ | C. pasteurianum protospacer 30 construct including 5′ | This study |
| | protospacer-adjacent sequence | |
| pSpacer30-3′ | C. pasteurianum protospacer 30 construct including 3′ | This study |
| | protospacer-adjacent sequence | |
| pSpacer30-flank | C. pasteurianum protospacer 30 construct including flanking | This study |
| | protospacer-adjacent sequence | |
| pCParray-cpaAIR | Type I-B CRISPR expression vector containing a synthetic | This study |
| | CRISPR array targeted to the C. pasteurianum cpaAIR gene | |
| pCParray-delcpaAIR | Type I-B CRISPR genome editing vector derived by inserting a | This study |
| | cpaAIR deletion editing cassette into pCParray-cpaAIR | |

TABLE 5
Oligonucleotides employed in this study

| Oligonucleotide | Sequence (5′-3′)* | SEQ ID NO. |
|---|---|---|
| Cas9.SacII.S | GTTTAGCCGCGGGGCAGCGCCTAAATGTAGAA | SEQ ID NO: 1 |
| Cas9.XhoI.AS | TCAGCTCTCGAGCAGTCTTGAAAAGCCCCTGTATTACTGC | SEQ ID NO: 2 |
| delcpaAIR.PvuI.S | CTACTACGATCGGTCCTAAAAGCAGGGTATGAAGTCCATTAG | SEQ ID NO: 3 |
| delcpaAIR.SOE.AS | CTTGAGGTCTAGGACTTCTATCTGGGAATAGAATGTTGTTCGATAGGCATC | SEQ ID NO: 4 |
| | C | |
| delcpaAIR.SOE.S | GGATGCCTATCGAACAACATTCTATTCCCAGATAGAAGTCCTAGACCTCAA | SEQ ID NO: 5 |
| | G | |
| delcpaAIR.PvuI.AS | GTCAAGCGATCGGCTTAGCTGGTAAGAAGCAAGGTCTT | SEQ ID NO: 6 |
| -cas9.SacII.S | GACGATCCGCGGGGTTACTTTTTATGGATAAGAAATACTCAATAGGC | SEQ ID NO: 7 |
| Cas9.BstZ17I.AS | CCTGTAGATAACAAATACGATTCTTCCGAC | SEQ ID NO: 8 |
| spacer18.AatII.S | GGTAAAATTTGATTGTCCTCATTGCGATGAAGAAAGACGT | SEQ ID NO: 9 |
| spacer18.SacII.AS | CTTTCTTCATCGCAATGAGGACAATCAAATTTTACCGC | SEQ ID NO: 10 |
| spacer24.AatII.S | GGTTGCAATAGAATGTGATAAAGACCATACTCATATGTGACGT | SEQ ID NO: 11 |
| spacer24.SacII.AS | CACATATGAGTATGGTCTTTATCACATTCTATTGCAACCGC | SEQ ID NO: 12 |
| spacer30.AatII.S | GGATAATATGGATTGAAGAGTGTTCAGAAGTTAAATAGACGT | SEQ ID NO: 13 |
| spacer30.SacII.AS | CTATTTAACTTCTGAACACTCTTCAATCCATATTATCCGC | SEQ ID NO: 14 |
| spacer18-5′.AatII.S | GGTTTCAGTAAAATTTGATTGTCCTCATTGCGATGAAGAAAGACGT | SEQ ID NO: 15 |
| spacer18-5′.SacII.AS | CTTTCTTCATCGCAATGAGGACAATCAAATTTTACTGAAACCGC | SEQ ID NO: 16 |
| spacer18-3′.AatII.S | GGGTAAAATTTGATTGTCCTCATTGCGATGAAGAAATAGAAAGACGT | SEQ ID NO: 17 |
| spacer18-3′.SacII.AS | CTTTCTATTTCTTCATCGCAATGAGGACAATCAAATTTTACCCGC | SEQ ID NO: 18 |
| spacer24-5′.AatII.S | GGAAATTGTTGCAATAGAATGTGATAAAGACCATACTCATATGTGACGT | SEQ ID NO: 19 |
| spacer24-5′.SacII.AS | CACATATGAGTATGGTCTTTATCACATTCTATTGCAACAATTTCCGC | SEQ ID NO: 20 |
| spacer24-3′.AatII.S | GGTTGCAATAGAATGTGATAAAGACCATACTCATATGTTTTTAAGACGT | SEQ ID NO: 21 |
| spacer24-3′.SacII.AS | CTTAAAAACATATGAGTATGGTCTTTATCACATTCTATTGCAACCGC | SEQ ID NO: 22 |
| spacer30-5′.AatII.S | GGTATCTATAATATGGATTGAAGAGTGTTCAGAAGTTAAATAGACGT | SEQ ID NO: 23 |
| spacer30-5′.SacII.AS | CTATTTAACTTCTGAACACTCTTCAATCCATATTATAGATACCGC | SEQ ID NO: 24 |
| spacer30-3′.AatII.S | GGATAATATGGATTGAAGAGTGTTCAGAAGTTAAATATGCTGGACGT | SEQ ID NO: 25 |
| spacer30-3′.SacII.AS | CCAGCATATTTAACTTCTGAACACTCTTCAATCCATATTATCCGC | SEQ ID NO: 26 |
| spacer18- | GGTTTCAGTAAAATTTGATTGTCCTCATTGCGATGAAGAAATAGAAAGACG | SEQ ID NO: 27 |
| flank.AatII.S | T | |
| spacer18- | CTTTCTATTTCTTCATCGCAATGAGGACAATCAAATTTTACTGAAACCGC | SEQ ID NO: 28 |
| flank.SacII.AS | | |
| spacer24- | GGAAATTGTTGCAATAGAATGTGATAAAGACCATACTCATATGTTTTTAAG | SEQ ID NO: 29 |
| flank.AatII.S | ACGT | |
| spacer24- | CTTAAAAACATATGAGTATGGTCTTTATCACATTCTATTGCAACAATTTCCG | SEQ ID NO: 30 |
| flank.SacII.AS | C | |
| spacer30- | GGTATCTATAATATGGATTGAAGAGTGTTCAGAAGTTAAATATGCTGGACG | SEQ ID NO: 31 |
| flank.AatII.S | T | |
| spacer30- | CCAGCATATTTAACTTCTGAACACTCTTCAATCCATATTATAGATACCGC | SEQ ID NO: 32 |
| flank.SacII.AS | | |
| cpaAIR.S | CATAACCTCAGCCATATAGCTTTTACCTACTCC | SEQ ID NO: 33 |
| cpaAIR.AS | ATAGGTGGATTCCCTTGTCAAGATTTTAGC | SEQ ID NO: 34 |

*Underline: restriction recognition sequence

### ABBREVIATIONS

CRISPR: Clustered Regularly Interspaced Short Palindromic Repeat; Cas: CRISPR-associated; PAM: protospacer adjacent motif; crRNA: CRISPR RNA; tracrRNA: trans-activating CRISPR RNA; gRNA: guide RNA; DN: DNA nick; DR: direct repeat; CFU: colony-forming unit; nt: nucleotide; cas68b753412: cas6-cas8b-cas7-cas5-cas3-cas4-cas1-cas2; DB: DNA break

### DETAILED DESCRIPTION OF INVENTION

Implementation of the Type II CRISPR-Cas9 System for Genome Editing in C. pasteurianum

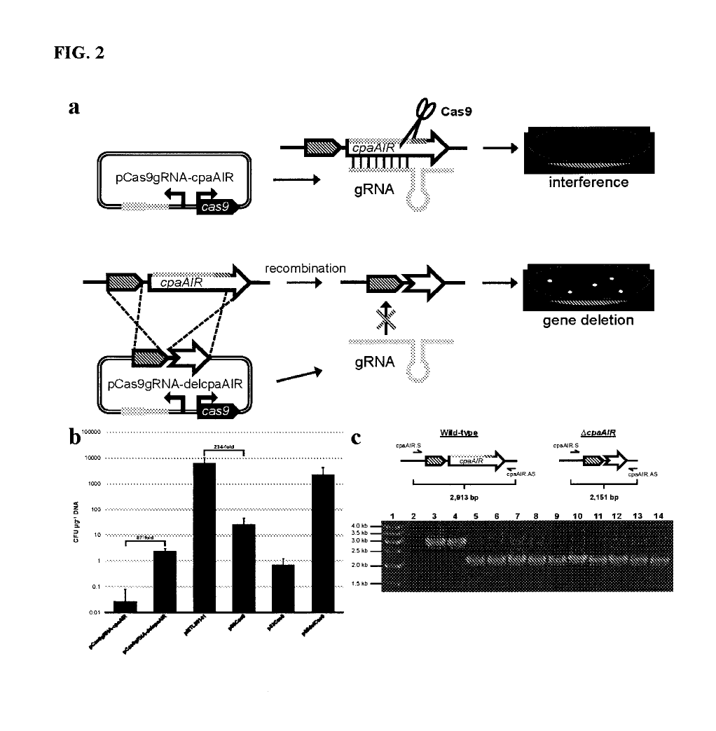

Recently, two groups reported a CRISPR-based methodology employing the Type II system from S. pyogenes for use in genome editing of C. beijerinckii and C. cellulolyticum (Wang, et al, 2015; Xu, et al, 2015). This system requires expression of the cas9 endonuclease gene in trans, in addition to a chimeric guide RNA (gRNA) containing a programmable RNA spacer. To determine if the S. pyogenes machinery could also function for genome editing in C. pasteurianum, we constructed a Type II CRISPR-Cas9 vector by placing cas9 under constitutive control of the C. pasteurianum thiolase (thl) gene promoter and designing a synthetic gRNA expressed from the C. beijerinckii sCbei_5830 small RNA promoter (Wang, et al, 2015). We selected the cpaAIR gene as a target double-stranded DB site through the use of a 20 nt spacer located within the cpaAIR coding sequence, as this gene has been previously disrupted in C. pasteurianum (Pyne, Moo-Young, et al, 2014). An S. pyogenes Type II PAM sequence (5′-NGG-3′), required for recognition and subsequent cleavage by Cas9 (Jiang, et al, 2013), is located at the 3′ end of the cpaAIR protospacer sequence within the genome of C. pasteurianum (FIG. 2A). Transformation of C. pasteurianum with the resulting vector, designated pCas9gRNA-cpaAIR, yielded an average transformation efficiency of 0.03 colony-forming units (CFU) μg⁻¹ DNA (FIG. 2B). Only one out of five attempts at transfer of pCas9gRNA-cpaAIR produced a single transformant, indicating efficient Cas9-mediated killing of host cells. To demonstrate genome editing using this system, we constructed pCas9gRNA-delcpaAIR through introduction of a cpaAIR gene deletion editing cassette into plasmid pCas9gRNA-cpaAIR. The editing cassette was designed to contain 1,029 bp and 1,057 bp homology regions to the cpaAIR locus, which together flank the putative cpaAIR double-stranded DB site. Homologous recombination between the plasm id-borne editing cassette and the C. pasteurianum chromosome is expected to result in a cpaAIR gene deletion comprising 567 bp of the cpaAIR coding sequence, including the protospacer and associated PAM element required for Cas9 attack, and 195 bp of the upstream cpaAIR gene region, including the putative cpaAIR gene promoter (FIG. 2A). Compared to the lethal pCas9gRNA-cpaAIR vector, introduction of pCas9gRNA-delcpaAIR established transformation. A transformation efficiency of 2.6 CFU μg⁻¹ DNA was obtained using pCas9gRNA-delcpaAIR, an 87-fold increase compared to pCas9gRNA-cpaAIR (FIG. 2B). Genotyping of 10 pCas9gRNA-delcpaAIR transformants generated the expected PCR product corresponding to cpaAIR gene deletion, resulting in an editing efficiency of 100% (FIG. 2C). Sanger sequencing of a single pCas9gRNA-delcpaAIR transformant confirmed successful deletion of a 762 bp region of the cpaAIR coding sequence (data not shown).

Despite an editing efficiency of 100% using heterologous Type II CRISPR-Cas9 machinery, an average of only 47 total CFU were obtained by introducing 15-25 pg of pCas9gRNA-delcpaAIR plasmid DNA (2.6 CFU μg⁻¹ DNA). Such a low transformation efficiency may impede more ambitious genome editing strategies, such as integration of large DNA constructs and multiplexed editing. Since expression of the Cas9 endonuclease has been shown to be moderately toxic in a multitude of organisms [e.g. mycobacteria, yeast, algae, and mice (Wang, et al, 2013; Jacobs, et al, 2014; Jiang, et al, 2014; Vandewalle, 2015)], even in the absence of a targeting gRNA, we prepared various cas9-expressing plasmid constructs to determine if expression of cas9 leads to reduced levels of transformation. Introduction of a cas9 expression cassette lacking a gRNA into plasmid pMTL85141 (transformation efficiency of 6.3×10³ CFU μg⁻¹ DNA), generating p85Cas9, resulted in a reduction in transformation efficiency of more than two orders of magnitude (26 CFU μg⁻¹ DNA) (FIG. 2B). Modifying the pIM13 replication module of p85Cas9 to one based on pCB102 (Heap, et al, 2009) in plasmid p83Cas9 further reduced transformation to barely detectable levels (0.7 CFU μg⁻¹ DNA). Importantly, transformation of C. pasteurianum with p85delCas9, constructed through deletion of the putative cas9 gene promoter in p85Cas9, restored transformation to typical levels (2.2×10³ CFU μg⁻¹ DNA). Collectively these data demonstrate that expression of Cas9 in the absence of a gRNA significantly reduces transformation of C. pasteurianum. It is noteworthy that we also observed a dramatically reduced level of transformation of Clostridium acetobutylicum using plasmid p85Cas9, which could also be rescued through deletion of the cas9 gene promoter in p85delCas9 (data not shown).

Analysis of the C. pasteurianum Type I-B CRISPR-Cas System and Identification of Putative Protospacer Matches to Host-Specified Spacers

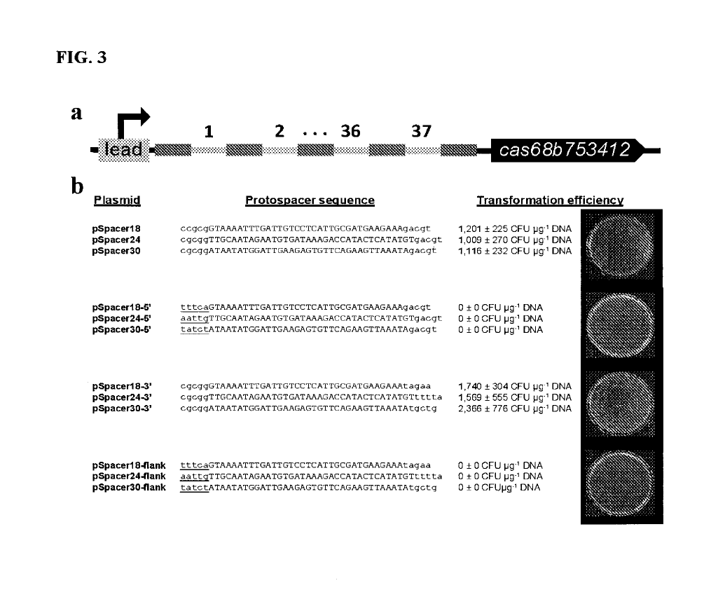

Due to the inhibitory effect of cas9 expression on transformation, we reasoned that the S. pyogenes Type II CRISPR-Cas9 system imposes significant limitations on genome editing in Clostridium, as the clostridia are transformed at substantially lower levels compared to most bacteria (Pyne, Bruder, et al, 2014). To evade poor transformation of cas9-encoded plasm ids, we investigated the prospect of genome editing using endogenous CRISPR-Cas machinery. We recently sequenced the genome of C. pasteurianum and unveiled a CRISPR-Cas system comprised of a 37-spacer CRISPR array upstream of a core cas gene operon (cas6-cas8b-cas7-cas5-cas3-cas4-casl-cas2) (FIG. 3A). An additional 8 spacers flanked by the same direct repeat sequence were found elsewhere in the genome, yet were not associated with putative Cas-encoding genes. The presence of cas3 and cas8b signature genes led to classification of this CRISPR-Cas locus within the Type I-B subtype.

We used BLAST (Altschul, et al, 1990) and PHAST (Zhou, et al, 2011) to analyze all 45 spacer tags specified in the C. pasteurianum genome in an attempt to identify protospacer matches from invading nucleic acid elements, including phages, prophages, plasm ids, and transposons. Since seed sequences, rather than full-length protospacers, have been shown to guide CRISPR interference (Semenova, et al, 2011), mismatches in the PAM-distal region of protospacer were permitted, while spacer-protospacer matches possessing more than one mismatch in 7 nt of PAM-proximal seed sequence were omitted. Although no perfect spacer-protospacer matches were identified, several hits were revealed possessing 2-7 mismatches to full-length C. pasteurianum spacers (Table 1). All protospacer hits identified were represented by spacers 18, 24, and 30 from the central C. pasteurianum Type I-B CRISPR array, whereby multiple protospacer hits were obtained using spacers 24 and 30. Importantly, protospacer matches were derived from predicted Clostridium and Bacillus phage and prophage elements.

Probing the C. pasteurianum Type I-B CRISPR-Cas System Using in Vivo Interference Assays and Elucidation of Protospacer Adjacent Motif (PAM) Sequences

We selected the best protospacer hits, possessing 2-4 nt mismatches to C. pasteurianum spacers 18, 24, and 30 (Table 1), for further characterization. Previous analyses of Type I CRISPR-Cas systems have employed a 5 nt mismatch threshold for identifying putative spacer-protospacer hits (Shah, et al, 2013; Gudbergsdottir, et al, 2011), as imperfect pairing affords flexibility in host recognition of invading elements or indicates evolution of invading protospacer sequences as a means of evading CRISPR attack (Semenova, et al, 2011). While the top spacer 30 hit was found to possess homology to an intact prophage from C. botulinum, the best spacer 24 match was predicted to target clostridial phage φCD111, a member of the Siphoviridae phage family. C. pasteurianum has recently been shown to harbor an intact and excisable temperate prophage from the same phage family, further supporting the notion that spacer 24 targets phage φCD111. The single protospacer match to spacer 18 was found to possess homology to a partial prophage region within the genome of C. pasteurianum BC1, a distinct strain from the type strain (ATCC 6013) employed in this study. Based on these analyses, it is probable that the phage and prophage elements described above are recognized by the C. pasteurianum Type I-B CRISPR-Cas machinery.

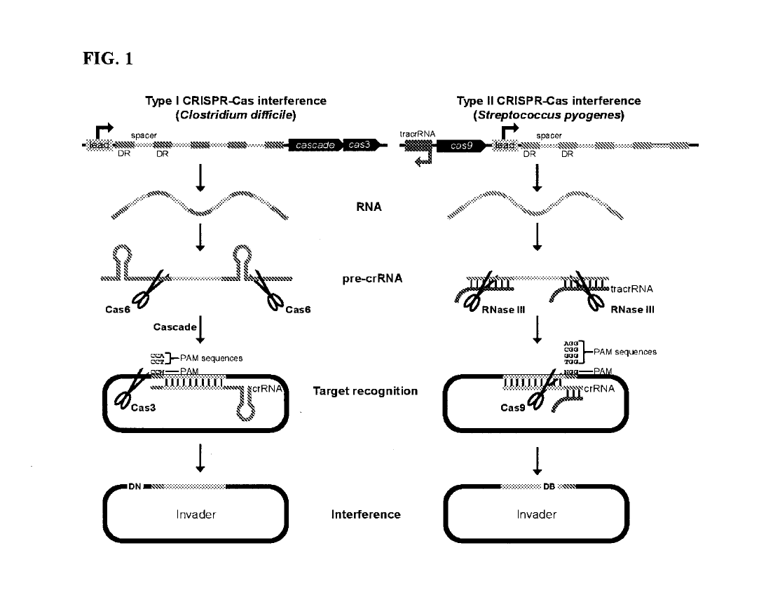

Spacers 18, 24, and 30 were utilized to assess activity of the C. pasteurianum Type I-B CRISPR-Cas system using plasmid transformation interference assays. C. pasteurianum spacer sequences, rather than the identified protospacer hits possessing 2-4 mismatches, were utilized as protospacers to ensure 100% identity between C. pasteurianum spacers and plasm id-borne protospacers. As Type I and II CRISPR-Cas systems require the presence of a PAM sequence for recognition of invading elements (Deveau, et al, 2008; Mojica, et al, 2009), a protospacer alone is not sufficient to elicit attack by host Cas proteins. Moreover, PAM elements are typically species-specific and vary in length, GC content, and degeneracy (Shah, et al, 2013). Accordingly, PAMs are often determined empirically and cannot be directly inferred from protospacer sequences. Hence, we constructed four derivatives each of protospacers 18, 24, and 30, yielding 12 constructs in total, whereby each protospacer was modified to contain different combinations of protospacer-adjacent sequence. Protospacer-adjacent sequences were derived from nucleotide sequences upstream or downstream of the protospacer matches within the DNA of the invading phage determinants depicted in Table 1. Five nt of protospacer-adjacent sequence was selected on the basis that most PAMs are encompassed within 5 nt (Shah, et al, 2013). Specifically, each protospacer derivative was constructed with one of four protospacer-adjacent sequence arrangements: 1) no protospacer-adjacent sequences; 2) 5 nt of 5′ protospacer-adjacent sequence; 3) 5 nt of 3′ protospacer-adjacent sequence; and 4) 5 nt of 5′ and 3′ protospacer-adjacent sequence (FIG. 3B). Although the PAM element is typically located at the 5′ end of protospacers in Type I CRISPR-Cas systems, which is opposite to the arrangement observed in Type II systems (Shah, et al, 2013) (FIG. 1), we elected to assay both 5′ and 3′ protospacer-adjacent sequences in the event that the C. pasteurianum Type I-B machinery exhibits atypical PAM recognition. Protospacer derivatives were synthesized as complementary single-stranded oligonucleotides, which were annealed and inserted into plasmid pMTL85141. Interestingly, all three protospacers triggered an interference response from C. pasteurianum when a suitable protospacer-adjacent sequence was provided (FIG. 3B). Plasmids devoid of 5′ protospacer-adjacent sequence (pSpacer18, pSpacer24, pSpacer30, pSpacer18-3′, pSpacer24-3′, and pSpacer30-3′), efficiently transformed C. pasteurianum (1.0-2.4×10³ CFU μg⁻¹ DNA) (FIG. 3B). Conversely, plasmids containing 5′ protospacer-adjacent sequence (pSpacer18-5′, pSpacer24-5′, pSpacer30-5′, pSpacer18-flank, pSpacer24-flank, and pSpacer30-flank), were unable to transform C. pasteurianum (FIG. 3B). These data indicate that C. pasteurianum expresses Cas proteins that recognize specific PAM sequences encompassed within 5 nt at the 5′ end of protospacers. Interference by host Cas proteins was found to be robust and highly specific.

We analyzed the 5′-adjacent sequences corresponding to protospacers 18, 24, and 30, resulting in three functional PAM sequences represented by 5′-TTTCA-3′, 5′-AATTG-3′, and 5′-TATCT-3′, respectively (FIG. 3B and Table 1). Due to the promiscuity of most PAM elements, the identified PAM sequences presumably represent only a small subset of sequences that together constitute the consensus recognized by C. pasteurianum. It is noteworthy, however, that the third nucleotide of all three functional PAM sequences, as well as six additional sequences that were not assayed in vivo (Table 1), represents a conserved thymine (T) residue, which may be essential for recognition of invading determinants by C. pasteurianum Cas proteins. Within protospacer constructs lacking 5′ adjacent sequence, namely pSpacer18, pSpacer24, pSpacer30, pSpacer18-3′, pSpacer24-3′, and pSpacer30-3′, protospacers are preceded by the sequence 5′-CCGCG-3′ or 5′-CGCGG-3′, encompassing the partial SacII cloning site. It is evident that this sequence does not constitute a PAM sequence recognized by C. pasteurianum CRISPR-Cas machinery (FIG. 3B). Similarly, in their native context within the chromosome of C. pasteurianum, spacers 18, 24, and 30 are preceded by the sequence 5′-TAAAT-3′, which is also not recognized by host Cas proteins in order to avoid self attack. Although this sequence resembles the three functional PAM sequences identified through interference assays, particularly 5′-TATCT-3′, the central conserved T nucleotide is lacking, further supporting the importance of this residue in self and non-self distinction by C. pasteurianum.

By assuming the PAM sequence recognized by C. pasteurianum is 5 nt in length and based on a C. pasteurianum chromosomal GC content of 30%, it is possible to calculate the frequency that each PAM sequence occurs within the genome of C. pasteurianum. All three 5 nt C. pasteurianum PAM sequences are comprised of four NT residues and one G/C residue, indicating that all PAM sequences should occur at the same frequency within the C. pasteurianum chromosome. Since the probability of an A or T nucleotide occurring in the genome is 0.35 and the probability of a C or G nucleotide is 0.15, the frequency of each PAM sequence within either strand of the C. pasteurianum genome is 1÷[(0.35)⁴(0.15)(2 strands)]=222 bp. More importantly, the overall PAM frequency is only 74 bp, indicating that one of the three functional PAM sequences is expected to occur every 74 bp within the genome of C. pasteurianum. This frequency is further reduced to 27 bp if the true PAM recognized by C. pasteurianum is represented by 3 nt, which is a common feature of Type I-B PAMs (Boudry, et al, 2015; Stoll, et al, 2013). In comparison, the Type II CRISPR-Cas9 system from S. pyogenes recognizes a 5′-NGG-3′ consensus, which is expected to occur every 22 bp in the genome of C. pasteurianum.

### Repurposing the Endogenous Type I-B CRISPR-Cas System for Markerless Genome Editing

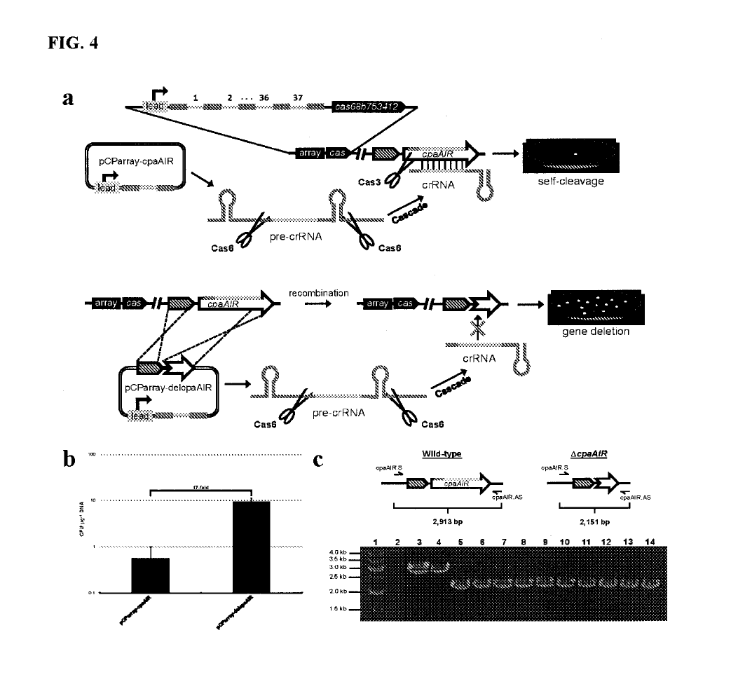

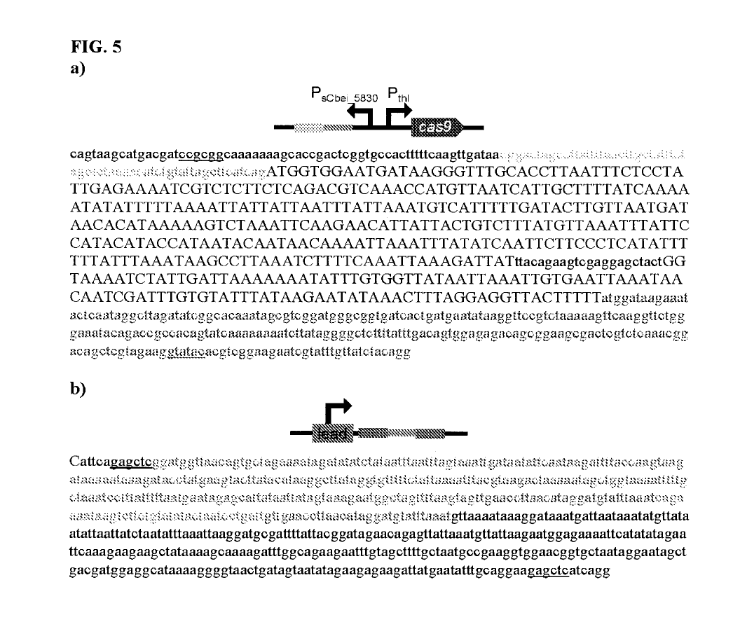

The high frequency of functional PAM sequences within the genome of C. pasteurianum suggests that the endogenous Type I-B CRISPR-Cas system could be co-opted to attack any site within the organism's chromosome and, therefore, provide selection against unmodified host cells. To first assess self-targeting of the C. pasteurianum CRISPR-Cas system, we again selected the cpaAIR gene as a target. The 891 bp cpaAIR gene was found to possess a total of 19 potential PAM sequences (5′-TTTCA-3′, 5′-AATTG-3′, and 5′-TATCT-3′), which is more than the 12 PAM sequences expected based on a genomic frequency of 74 bp. We selected one PAM sequence (5′-AATTG-3′) within the coding region of the cpaAIR gene as the target site for C. pasteurianum self-cleavage, whereby sequence immediately downstream embodies the target protospacer. Analysis of the core 37 spacers encoded by C. pasteurianum revealed minimal variation in spacer length (34-37 nt; mean of 36 nt), while GC content was found to vary dramatically (17-44%). Subsequently, we generated a synthetic cpaAIR spacer by selecting 36 nt immediately downstream of the designated PAM sequence, which was found to possess a GC content of 28%. A CRISPR expression cassette was designed by mimicking the sequence and arrangement of the native Type I-B CRISPR array present in the C. pasteurianum genome (FIG. 5B). Specifically, a 243 bp CRISPR leader was utilized to drive transcription of the synthetic cpaAIR CRISPR array, comprised of the 36 nt cpaAIR spacer flanked by 30 nt direct repeats. The synthetic array was followed by 298 bp of sequence located at the 3′ end of the endogenous chromosomal CRISPR array. The resulting cassette was synthesized and inserted into plasmid pMTL85141, generating pCParray-cpaAIR (FIG. 4A). While several attempts at transformation of C. pasteurianum using pCParray-cpaAIR failed to generate transformants, an overall transformation efficiency of 0.6 CFU μg⁻¹ DNA was obtained (FIG. 4B), compared to 6.3×10³ CFU μg⁻¹ DNA for the pMTL85141 parental plasmid, a difference of more than four orders of magnitude. We reasoned that the synthetic cpaAIR spacer triggered self-attack of C. pasteurianum through introduction of a DN and subsequent strand degradation by Cas3. To verify the location of the DN site within the cpaAIR target gene and, more importantly, demonstrate manipulation of the Type I-B CRISPR-Cas system for genome editing, we introduced the aforementioned cpaAIR editing cassette utilized for cas9-mediated genome editing (from plasmid pCas9gRNA-delcpaAIR) into plasmid pCParray-cpaAIR (FIG. 4A). Transformation of C. pasteurianum with the resulting plasmid, pCParray-delcpaAIR, produced an abundance of transformants, yielding a transformation efficiency of 9.5 CFU μg⁻¹ DNA, an increase of more than an order of magnitude compared to pCParray-cpaAIR lacking an editing cassette (FIG. 4B). Despite a low-level of background resulting from transformation with pCParray-cpaAIR, genotyping of 10 pCParray-delcpaAIR transformants generated a PCR product corresponding to cpaAIR gene deletion in all colonies screened, yielding an editing efficiency of 100% (FIG. 4C). Sanger sequencing of a single pCParray-delcpaAIR transformant confirmed successful deletion of a 762 bp region of the cpaAIR coding sequence (data not shown). Importantly, this outcome is consistent with localization of the DN within the cpaAIR locus, as well as provides proof-of-principle repurposing of the host Type I-B CRISPR-Cas machinery for efficient markerless genome editing.

### Identification of Putative PAM Sequences in Industrial and Pathogenic Clostridia

As the first step towards expanding our CRISPR-Cas hijacking strategy to other prokaryotes, we surveyed the clostridia for species harboring putative CRISPR-Cas loci. One cellulolytic and one acetogenic species, namely Clostridium thermocellum and Clostridium autoethanogenum, respectively, in addition to Clostridium tetani, a human pathogen, were selected. Like C. pasteurianum, all three species encode putative Type I-B systems, while C. tetani (Brüggemann, et al, 2015) and C. thermocellum (Brown, et al, 2014) harbor an additional Type I-A or Type III locus, respectively. Only spacers associated with Type I-B loci were analyzed, corresponding to 98, 31, and 169 spacers from C. autoethanogenum, C. tetani, and C. thermocellum, respectively. In silico analysis of clostridial spacers against firm icute genomes, phages, and plasm ids yielded putative protospacer matches from all three clostridial Type I-B CRISPR-Cas loci analyzed (Table 2). In total 10 promising protospacer hits were obtained, which were found to target phages (2 hits), plasmids (1 hit), predicted prophages (5 hits), and regions of bacterial genomes in the vicinity of phage and/or transposase genes (2 hits). Six spacers were found to target clostridial genomes and clostridial phage and prophage elements. Interestingly, spacers from the C. autoethanogenum Type I-B locus were analyzed in an earlier report and no putative protospacer matches were identified (Brown, et al, 2014), whereas we unveiled four probable protospacer hits, including the only perfect spacer-protospacer match identified in this study. Overall, putative protospacer matches contained 0-8 mismatches when aligned with clostridial spacers. Analysis of clostridial 5′-protospacer-adjacent sequences revealed a number of conserved sequences (Table 2). Interestingly, all 10 putative PAM sequences were found to possess a conserved A residue in the immediate 5′ protospacer-adjacent position. Based on a 3 nt consensus, prospective PAMs of 5′-NAA-3′ (PAM sequences: 5′-CAA-3′, 5′-GAA-3′, 5′-TAA-3′, and 5′-TAA-3′), 5′-TNA-3′ (PAM sequences: 5′-TAA-3′, 5′-TCA-3′, and 5′-TTA-3′), and 5′-NCA-3′ (PAM sequences: 5′-ACA-3′, 5′-TCA-3′, 5′-TCA-3′) could be predicted for the Type I-B CRISPR-Cas loci of C. autoethanogenum, C. tetani, and C. thermocellum, respectively.

### Discussion

This invention details the development of a genome editing methodology allowing efficient introduction of precise chromosomal modifications through harnessing an endogenous CRISPR-Cas system. Our strategy leverages the widespread abundance of prokaryotic CRISPR-Cas machinery, which have been identified in 45% of bacteria, including 74% of clostridia (Grissa, et al, 2007). An exceptional abundance of CRISPR-Cas loci, coupled with an overall lack of sophisticated genetic engineering technologies and tremendous biotechnological potential, provides the rationale for our proposed genome editing strategy in Clostridium. We selected C. pasteurianum for proof-of-concept CRISPR-Cas repurposing due to the presence of a Type I-B CRISPR-Cas locus (FIG. 3A) and established industrial relevance for biofuel production (Johnson, et al, 2007; Yazdani, 2007). Analysis of C. pasteurianum CRISPR tags led to elucidation of the probable origins of three spacer sequences, all of which returned protospacer matches from clostridial phage and prophage determinants (Table 1). C. pasteurianum Cas proteins proved to be functional and highly active against plasmid-borne protospacers possessing a 5′ adjacent PAM sequence, as no interference response was generated from protospacers harboring 3′ adjacent sequence in the absence of a 5′ PAM sequence (FIG. 3B). This finding is consistent with other Type I CRISPR-Cas systems, in which the PAM positioned 5′ to the protospacer is essential for interference by host cells and contrasts Type II CRISPR-Cas9 systems, whereby the PAM is recognized at the 3′ end of protospacers (Barrangou, et al, 2007; Mojica, et al, 2009; Shah, et al, 2013). Following elucidation of functional PAM sequences, we developed a genome editing strategy encompassing expression of a synthetic programmable Type I-B CRISPR array that guides site-specific nucleolytic attack of the C. pasteurianum chromosome by co-opting the organism's native Cas proteins. Cas3-mediated DNA attack affords selection against unmodified host cells, whereby edited cells are efficiently obtained through co-introduction of an editing template (FIG. 4A, B). We have demonstrated 100% editing efficiency (10/10 correct colonies) by targeting the cpaAIR locus in combination with introduction of a cpaAIR gene deletion cassette (FIG. 4C).

Our native CRISPR-Cas repurposing methodology contrasts current approaches of CRISPR-mediated genome editing in bacteria, which rely on the widely-employed Type II CRISPR-Cas9 system from S. pyogenes. In Clostridium, such heterologous CRISPR-Cas9 genome editing strategies have recently been implemented in C. beijerinckii (Wang, et al, 2015) and C. cellulolyticum (Xu, et al, 2015). While editing efficiencies >95% were reported using C. cellulolyticum, no efficiency was provided for CRISPR-based editing in C. beijerinckii, which involves the use of a phenotypic screen to identify mutated cells (Wang, et al, 2015). Although we have shown 100% editing efficiency in C. pasteurianum through application of the same S. pyogenes CRISPR-Cas9 machinery (FIG. 2A, C), the total yield of edited cells was only 25% compared to the endogenous Type I-B CRISPR-Cas approach (FIG. 2B and 4B). By assessing transformation of various cas9 expression constructs, we ascribe this outcome to poor transformation of vectors expressing cas9 in trans (FIG. 2B). A low to moderate level of Cas9 toxicity has been documented in a diverse range of organisms, including protozoa (Peng, et al, 2015), Drosophila (Gratz, et al, 2014; Sebo, et al, 2014), yeast (Jacobs, et al, 2014), mice (Wang, et al, 2013), and human cells (Charpentier, 2013), and likely results from the generation of lethal ectopic chromosomal DNA breaks. We have also observed reduced transformation of E. coli ER1821 in this study using plasmids expressing heterologous cas9 (data not shown). In more dramatic instances, for example in mycobacteria (Vandewalle, 2015) and the alga Chlamydomonas reinhardtii (Jiang, et al, 2014), toxicity leads to erratic cas9 expression and overall poor genome editing outcomes. Such reports emphasize the importance of mitigating Cas9 toxicity or developing alternative methodologies facilitating efficient genome editing (Jiang, et al, 2014). Owing to the notoriously low transformation efficiencies achieved using Clostridium species (typically 10²-10³ CFU μg⁻¹ DNA) (Pyne, Bruder, et al, 2014), the clostridia are especially susceptible to the detrimental effects of heterologous cas9 expression, as observed in this study. Hence, for key organisms lacking endogenous CRISPR-Cas loci, such as C. acetobutylicum and C. ljungdahlii, in which the heterologous Type II system is obligatory for genome editing, we recommend inducible expression of cas9. For this purpose, several clostridial inducible gene expression systems have recently been characterized (Dong, et al, 2012; Hartman, et al, 2011). Our success in obtaining targeted mutants using constitutive expression of heterologous cas9 potentially results from the relatively high efficiency of plasmid transfer to C. pasteurianum (up to 10⁴ CFU μg⁻¹ DNA) (Pyne, et al, 2013). It is probable that Cas9-mediated genome editing efforts could be impeded in species that are poorly transformed, rendering endogenous CRISPR-Cas machinery the preferred platform for genome editing. Furthermore, since linear DNA is a poor substrate for transformation of Clostridium and because it is generally unfeasible to co-transfer two DNA substrates to Clostridium due to poor transformation, all of the genetic components required for Type I-B or Type II CRISPR-Cas functionality in this study were expressed from single vectors. This shortcoming exposes an additional advantage of our endogenous CRISPR-Cas hijacking strategy, as only a small CRISPR array (0.6 kb) and editing template are required for genome editing, resulting in a compact 5.7 kb editing vector (pCParray-delcpaAIR). On the other hand, editing using the heterologous Type II system requires expression of the large 4.2 kb cas9 gene, in addition to a 0.4 kb gRNA cassette and editing template. The large size of the resulting pCas9gRNA-delcpaAIR editing vector (9.7 kb) not only limits transformation but also places significant constraints on multiplexed editing strategies involving multiple gRNAs and editing templates. Owing to overall low rates of homologous recombination in Clostridium, such ambitious genome editing strategies could be enhanced through coupling of native or heterologous CRISPR-Cas machinery to highly recombinogenic phage activities (Datta, et al, 2008). In this context, one functional clostridial phage recombinase has been characterized to date (Dong, et al, 2014).

To initiate efforts aimed at co-opting Type I CRISPR-Cas machinery in other key species, we examined CRISPR spacer tags from one acetogenic (C. autoethanogenum), one cellulolytic (C. thermocellum), and one pathogenic (C. tetani) species (Table 2). Subsequent in silico analysis of clostridial spacers, coupled with our experimental validation of C. pasteurianum PAM sequences and a recent report detailing characterization of the C. difficile Type I-B CRISPR-Cas locus (Boudry, et al, 2015), provide an in depth glimpse into clostridial CRISPR-Cas defence mechanisms (Table 3). Overall, clostridial Type I-B PAM sequences are characterized by a notable lack of guanine (G) residues. Additionally, several PAM sequences unveiled in this study are recognized across multiple species of Clostridium, such as 5′-TCA-3′ by C. pasteurianum, C. tetani, and C. thermocellum, and 5′-TAA-3′ by C. autoethanogenum and C. tetani, which suggests horizontal transfer of CRISPR-Cas loci between these organisms. Indeed, C. tetani harbors 7 distinct Type I-B CRISPR arrays (Brüggemann, et al, 2015), 3 of which employ the same direct repeat sequence utilized by the C. pasteurianum Type I-B system. Since PAM sequences determined in this study are highly similar between C. pasteurianum (5′-TCA-3′, 5′-TTG-3′, 5′-TCT-3′) and C. tetani (5′-TCA-3′, 5′-TTA-3′, 5′-TAA-3′), it is plausible that these organisms recognize the same PAM consensus. More broadly, clostridial Type I-B PAM sequences bear a striking overall resemblance to sequences recognized by the Type I-B system from the distant archaeon Haloferax volcanii (5′-ACT-3′, 5′-TTC-3′, 5′-TAA-3′, 5′-TAT-3′, 5′-TAG-3′, and 5′-CAC-3′) (Stoll, et al, 2013), which are also distinguished by an overall low frequency of G residues. Collectively these data suggest that many PAM sequences are common amongst Type I-B CRISPR-Cas systems, even in evolutionarily distant species, such as the case of Haloferax and Clostridium. In this context, we posit that empirical elucidation of PAMs is unnecessary, as highly pervasive PAM sequences (e.g., 5′-TCA-3′ and 5′-TAA-3′) or validated sequences from closely-related species can easily be assessed for functionality in a target host strain. This consequence simplifies our proposed CRISPR-Cas repurposing approach, as a functional PAM sequence and a procedure for plasmid transformation are the only prerequisite criteria for implementing our methodology in any target organism harboring active Type I CRISPR-Cas machinery.

Genome editing strategies based on the S. pyogenes Type II system reported previously (Wang, et al, 2015; Xu, et al, 2015) and the CRISPR-Cas hijacking approach detailed in this study, represent a key divergence from earlier methods of gene disruption and integration in Clostridium (Pyne, Bruder, et al, 2014). Currently, the only procedures validated for modifying the genome of C. pasteurianum involve the use of a programmable group II intron (Pyne, Moo-Young, et al, 2014) and heterologous counter-selectable mazF marker (Sandoval, et al, 2015). Whereas group II introns are limited to gene disruption, as deletion and replacement are not possible, techniques based on homologous recombination using antibiotic resistance determinants and counter-selectable markers, such as pyrEIpyrF, codA, and mazF (Al-Hinai, et al, 2012; Heap, et al, 2012; Cartman, et al, 2012), are technically-challenging and laborious due to a requirement for excision and recycling of markers. In general, these strategies do not provide adequate selection against unmodified cells, necessitating subsequent rounds of enrichment and selection (Al-Hinai, et al, 2012; Heap, et al, 2012; Cartman, et al, 2012; Olson, 2012). Thus, both native and heterologous CRISPR-Cas machineries offer more robust platforms for genome modification of C. pasteurianum and related clostridia.

Currently, endogenous CRISPR-Cas systems have been harnessed in only a few prokaryotes, namely E. coli (Gomaa, et al, 2014; Luo, Mullis, et al, 2015), Pectobacterium atrosepticum (Vercoe, et al, 2013), Streptococcus thermophiles (Gomaa, et al, 2014), and two species of archaea (Li, et al, Nucleic Acids Res, 2015; Zebec, et al, 2014). In conjunction with these reports, our success in co-opting the chief C. pasteurianum CRISPR-Cas locus contributes to a growing motivation towards harnessing host CRISPR-Cas machinery in a plethora of prokaryotes. The general rationale of endogenous CRISPR-Cas repurposing is not limited to genome editing, as a range of applications can be envisioned. In a recent example, Luo et al. (Luo, Mullis, et al, 2015) deleted the native cas3 endonuclease gene from E. coli, effectively converting the host Type I-E CRISPR-Cas immune system into a robust transcriptional regulator for gene silencing. Such applications dramatically extend the existing molecular genetic toolbox and pave the way to advanced strain engineering technologies. Although our work here focused on C. pasteurianum, repurposing of endogenous CRISPR-Cas loci is readily adaptable to most of the genus Clostridium, including many species of immense relevance to medicine, energy, and biotechnology, as well as half of all bacteria and most archaea.

### EXAMPLES

The following examples are provided by way of illustration and not by limitation.

### Example 1

### Strains, Plasmids, and Oligonucleotides

Strains and plasm ids employed in this study are listed in Table 4. Clostridium pasteurianum ATCC 6013 was obtained from the American Type Culture Collection (ATCC; Manassas, Va.) and propagated and maintained according to previous methods (Pyne, et al, 2013; Pyne, Moo-Young, et al, 2014). Escherichia coli strains DH5α and ER1821 (New England Biolabs; Ipswich, Mass.) were employed for plasmid construction and plasmid methylation, respectively. Recombinant strains of C. pasteurianum were selected using 10 μg ml⁻¹ thiamphenicol and recombinant E. coli cells were selected using 30 μg ml⁻¹ kanamycin or 30 μg ml⁻¹ chloramphenicol. Antibiotic concentrations were reduced by 50% for selection of double plasmid recombinant cells. Desalted oligonucleotides and synthetic DNA constructs were purchased from Integrated DNA Technologies (IDT; Coralville, Iowa). Oligonucleotides utilized in this study are listed in Table 5 and synthetic DNA constructs are detailed in FIG. 5.

### Example 2

### DNA Manipulation, Plasmid Construction, and Transformation

A cas9 E. coli-Clostridium expression vector, p85Cas9, was constructed through amplification of a cas9 gene cassette from pCas9 (Jiang, et al, 2015) using primers cas9.SacII.S (SEQ ID NO 1)+cas9.Xhol.AS (SEQ ID NO 2) and insertion into the corresponding sites of pMTL85141 (Heap, et al, 2009). To construct an E. coli-C. pasteurianum Type II CRISPR-Cas9 plasmid (pCas9gRNA-cpaAIR) based on the S. pyogenes CRISPR-Cas9 system, we designed a synthetic gRNA cassette targeted to the C. pasteurianum cpaAIR gene by specifying a 20 nt cpaAIR spacer sequence (ctgatgaagctaatacagat, SEQ ID NO 36), which was expressed from the C. beijerinckii sCbei_5830 small RNA promoter (Wang, et al, 2015; SEQ ID NO 38). A promoter from the C. pasteurianum thiolase gene (SEQ ID NO 39) was included for expression of cas9. The resulting 821 bp DNA fragment (FIG. 5A; SEQ ID NO 35) was synthesized and inserted into the SacII and BstZ17I sites of p85Cas9. To modify pCas9gRNA-cpaAIR for genome editing via deletion of cpaAIR, splicing by overlap extension (SOE) PCR was utilized to fuse 1,028 bp and 1,057 bp cpaAIR homology regions generated using the primer sets delcpaAIR.Pvul.S (SEQ ID NO 3) +delcpaAIR.SOE.AS (SEQ ID NO 4) and delcpaAIR.SOE.S (SEQ ID NO 5) +delcpaAIR.Pvul.AS (SEQ ID NO 6), respectively. The resulting Pvul-digested product was cloned into the Pvul site of pCas9gRNA-cpaAIR, yielding pCas9gRNA-delcpaAIR. Plasmid p83Cas9, a p85Cas9 derivative containing the pCB102 replication module (Heap, et al, 2009), was constructed by amplifying cas9 from pCas9 (Jiang, et al, 2013) using primers cas9.SacII.S (SEQ ID NO 1) +cas9.Xhol.AS (SEQ ID NO 2) and inserting the resulting product into the corresponding sites of pMTL83151 (Heap, et al, 2009). A promoterless cas9 derivative of p85Cas9, designated p85delCas9, was derived by amplification of a partial promoterless cas9 fragment from pCas9gRNA-cpaAIR using primers -cas9.SacII.S (SEQ ID NO 7)+cas9.BstZ17I.AS (SEQ ID NO 8) and cloning of the resulting product into the SacII +BstZ17I sites of p85Cas9.

C. pasteurianum protospacer constructs lacking protospacer-adjacent sequences were derived by annealing oligos spacerl8.AatII.S (SEQ ID NO 9)+spacerl8.SacII.AS (SEQ ID NO 10) (pSpacer18), spacer24.AatILS (SEQ ID NO 11)+spacer24.SacII.AS (SEQ ID NO 12) (pSpacer24), or spacer30.AatILS (SEQ ID NO 13)+spacer30.SacILAS (SEQ ID NO 14) (pSpacer30). Protospacer constructs possessing 5′ or 3′ protospacer-adjacent sequences were prepared by annealing oligos spacer18-5′.AatII.S (SEQ ID NO 15) +spacer18-5′.SacII.AS (SEQ ID NO 16) (pSpacer18-5′), spacer18-3′.AatILS (SEQ ID NO 17) +spacer18-3′.SacII.AS (SEQ ID NO 18) (pSpacer18-3′), spacer24-5′.AatILS (SEQ ID NO 19) +spacer24-5′.SacII.AS (SEQ ID NO 20) (pSpacer24-5′), spacer24-3′.AatILS (SEQ ID NO 21) +spacer24-3′.SacII.AS (SEQ ID NO 22) (pSpacer24-3′), spacer30-5′.AatIl.S (SEQ ID NO 23) +spacer30-5′.SacII.AS (SEQ ID NO 24) (pSpacer30-5′), or spacer30-3′.AatIl.S (SEQ ID NO 25) +spacer30-3′.SacII.AS (SEQ ID NO 26) (pSpacer30-3′). Protospacer constructs possessing 5′ and 3′ flanking protospacer-adjacent sequence were prepared by annealing oligos spacer18-flank.AatILS (SEQ ID NO 27) +spacer18-flank.SacII.AS (SEQ ID NO 28) (pSpacer18-flank), spacer24-flank.AatILS (SEQ ID NO 29) +spacer24-flank.SacII.AS (SEQ ID NO 30) (pSpacer24-flank), or spacer30-flank.AatILS (SEQ ID NO 31) +spacer30-flank.SacILAS (SEQ ID NO 32) (pSpacer30-flank). In all instances protospacer oligos were designed such that annealing generated Aatll and Sacll cohesive ends for ligation with Aatll- +Sacll-digested pMTL85141.

To construct the endogenous CRISPR array vector, pCParray-cpaAIR, a synthetic CRISPR array was designed containing a 243 bp CRISPR leader sequence (SEQ ID NO 44) and a 37 nt cpaAIR spacer (SEQ ID NO 42) flanked by 30 nt direct repeat (SEQ ID NO 43) sequences. The synthetic array was followed by 298 bp of sequence (SEQ ID NO 56) found downstream of the endogenous CRISPR array in the chromosome of C. pasteurianum to ensure design of the synthetic array mimics that of the native sequence. The resulting 667 bp fragment (FIG. 5B, SEQ ID NO 41) was synthesized and cloned into the Sac! site of pMTL85141. A genome editing derivative of pCParray-cpaAIR for deletion of cpaAIR was derived by subcloning the Pvul-flanked cpaAIR deletion cassette from pCas9gRNA-delcpaAIR into pCParray-cpaAIR, yielding pCParray-delcpaAIR.

DNA manipulation was performed according to established methods (Sambrook, et al, 1989). Commercial kits for DNA purification and agarose gel extraction were obtained from Bio Basic Inc. (Markham, ON). Plasmids were introduced to C. pasteurianum (Pyne, et al, 2013) and E. coli (Sambrook, et al, 1989) using established methods of electrotransformation. Prior to transformation of C. pasteurianum, E. coli-C. pasteurianum shuttle plasm ids were first methylated in E. coli ER1821 by the M.FnuDII methyltransferase from plasmid pFnuDIIMKn (Pyne, Moo-Young, et al, 2014). One to 5 pg of plasmid DNA was utilized for transformation of C. pasteurianum, except for plasm ids harbouring CRISPR-Cas machinery (pCas9gRNA-cpaAIR, pCas9gRNA-delcpaAIR, pCParray-cpaAIR, and pCParray-delcpaAIR), in which 15-25 pg was utilized to enhance transformation. Transformation efficiencies reported represent averages of at least two independent experiments and are expressed as colony-forming units (CFU) per pg of plasmid DNA.

### Example 3

Identification of Putative Protospacer Matches to clostridial Spacers

Clostridial spacers were utilized to query firmicute genomes, phages, transposons, and plasm ids using BLAST. Parameters were optimized for somewhat similar sequences (BlastN) (Altschul, et al, 1990). Putative protospacer hits were assessed based on the number and location of mismatches, whereby multiple PAM-distal mutations were tolerated, while protospacers containing more than one mismatch within 7 nt of PAM-proximal seed sequence were rejected (Semenova, et al, 2011). Firm icute genomes possessing putative protospacer hits were analyzed for prophage content using PHAST (Zhou, et al, 2011) and surrounding sequences were inspected for elements indicative of DNA mobility and invasion, such as transposons, transposases, integrases, and term inases.

We claim:

1. A method for making site-specific changes to the genome of the bacterium Clostridium pasteurianum.

2. The method of claim 1 wherein said method involves the use of the cas9 enzyme of Streptococcus pyogenes.

3. The method of claim 1 wherein said method involves the use of one or more contiguous DNA sequences from the genome of Clostridium pasteurianum, wherein said one or more DNA sequences are repetitive sequences associated with the endogenous Clustered Regularly Interspersed Short Palindromic Repeats (CRISPR) system of Clostridium pasteurianum.

4. The method of claim 3 wherein said contiguous DNA sequence is the DNA sequence of SEQ ID NO 43.

5. The method of claim 3 wherein said contiguous DNA sequence is the DNA sequence of SEQ ID NO 45.

6. The method of claim 3 wherein said method involves the use of one or more contiguous DNA sequences from the native or modified genome of Clostridium pasteurianum, wherein said one or more contiguous DNA sequences is present in the native or modified genome of Clostridium pasteurianum immediately following to the 3′ side of a 5 nucleotide-long continuous sequence of DNA, commonly known to one versed in the art of CRISPR tools as a ‘protospacer adjacent motif’, wherein said 5 nucleotide-long continuous sequence of DNA is selected from the group consisting of 5′-TTTCA-3′, 5′-AATTG-3′, and 5′-TATCT-3′.

7. The method of claim 3 wherein said method involves the use of one or more contiguous DNA sequences from the native or modified genome of Clostridium pasteurianum, wherein said one or more contiguous DNA sequences is present in the native or modified genome of Clostridium pasteurianum immediately following to the 3′ side of a 5 nucleotide-long continuous sequence of DNA, commonly known to one versed in the art of CRISPR tools as a ‘protospacer adjacent motif’, wherein said 5 nucleotide-long continuous sequence of DNA is selected from the group consisting of 5′-AATTA-3′, 5′-AATTT-3′, 5′-TTTCT-3′, 5′-TCTCA-3′, 5′-TCTCG-3′, and 5′-TTTCA-3′.

8. The method of claim 3 wherein said method involves the use of one or more contiguous DNA sequences from the native or modified genome of Clostridium pasteurianum, wherein said one or more contiguous DNA sequences is present in the native or modified genome of Clostridium pasteurianum immediately following to the 3′ side of a 3 nucleotide-long continuous sequence of DNA, commonly known to one versed in the art of CRISPR tools as a ‘protospacer adjacent motif’, wherein said 3 nucleotide-long continuous sequence of DNA is selected from the group consisting of 5′-TCA-3′, 5′-TTG-3′, and 5′-TCT-3′.

9. A Clostridium pasteurianum bacterial cell whose genome has been altered through the use of the method of claim 2.

10. A Clostridium pasteurianum bacterial cell whose genome has been altered through the use of the method of claim 3.

11. A Clostridium pasteurianum bacterial cell whose genome has been altered through the use of the method of claim 4.

12. A Clostridium pasteurianum bacterial cell whose genome has been altered through the use of the method of claim 5.

13. A Clostridium pasteurianum bacterial cell whose genome has been altered through the use of the method of claim 6.

14. A Clostridium pasteurianum bacterial cell whose genome has been altered through the use of the method of claim 7.

15. A Clostridium pasteurianum bacterial cell whose genome has been altered through the use of the method of claim 8.

16. A method for making site-specific changes the genome of a bacterial cell selected from the group consisting of Clostridium autoethanogenum, Clostridium tetani, and Clostridium thermocellum.

17. The method of claim 16 wherein said method involves the use of one or more contiguous DNA sequences from the genome of bacterial cell whose genome is being changed, wherein said one or more DNA sequences are repetitive sequences associated with the endogenous Clustered Regularly Interspersed Short Palindromic Repeats (CRISPR) of said bacterial cell.

18. The method of claim 17 wherein said bacterial cell is Clostridium autoethanogenum and said one or more DNA sequences are selected from the group consisting of SEQ ID NO: 46 and SEQ ID NO: 47.

19. The method of claim 17 wherein said bacterial cell is Clostridium tetani and said one or more DNA sequences are selected from the group consisting of SEQ ID NO: 48, SEQ ID NO: 49, and SEQ ID NO: 50.

20. The method of claim 17 wherein said bacterial cell is Clostridium thermocellum one or more DNA sequences are selected from the group consisting of SEQ ID NO: 51, SEQ ID NO: 52, and SEQ ID NO: 53.

21. The method of claim 17 wherein said bacterial cell is Clostridium autoethangenum and said method involves the use of one or more contiguous DNA sequences from the native or modified genome of a Clostridium autoethangenum, wherein said one or more contiguous DNA sequences is present in the native or modified genome of Clostridium autoethangenum immediately following to the 3′ side of a 5 nucleotide-long continuous sequence of DNA, commonly known to one versed in the art of CRISPR tools as a ‘protospacer adjacent motif’, wherein said 5 nucleotide-long continuous sequence of DNA is selected from the group consisting of 5′-ATTAA-3′, 5′-ACTAA-3′, 5′-AAGAA-3′, and 5′-ATCAA-3′.

22. The method of claim 17 wherein said bacterial cell is Clostridium autoethangenum and said method involves the use of one or more contiguous DNA sequences from the native or modified genome of a Clostridium autoethangenum, wherein said one or more contiguous DNA sequences is present in the native or modified genome of Clostridium autoethangenum immediately following to the 3′ side of a 3 nucleotide-long continuous sequence of DNA, commonly known to one versed in the art of CRISPR tools as a ‘protospacer adjacent motif’, wherein said 3 nucleotide-long continuous sequence of DNA is 5′-NAA-3′, where ‘N’ is a nucleotide selected from the group consisting of ‘A’, ‘C’, ‘G’, and ‘T’.

23. The method of claim 17 wherein said bacterial cell is Clostridium tetani and said method involves the use of one or more contiguous DNA sequences from the native or modified genome of a Clostridium tetani, wherein said one or more contiguous DNA sequences is present in the native or modified genome of Clostridium tetani immediately following to the 3′ side of a 5 nucleotide-long continuous sequence of DNA, commonly known to one versed in the art of CRISPR tools as a ‘protospacer adjacent motif’, wherein said 5 nucleotide-long continuous sequence of DNA is selected from the group consisting of 5′-TTTTA-3′, 5′-TATAA-3′, and 5′-CATCA-3′.

24. The method of claim 17 wherein said bacterial cell is Clostridium tetani and said method involves the use of one or more contiguous DNA sequences from the native or modified genome of a Clostridium tetani, wherein said one or more contiguous DNA sequences is present in the native or modified genome of Clostridium tetani immediately following to the 3′ side of a 3 nucleotide-long continuous sequence of DNA, commonly known to one versed in the art of CRISPR tools as a ‘protospacer adjacent motif’, wherein said 3 nucleotide-long continuous sequence of DNA is 5′-TNA-3′, where ‘N’ is a nucleotide selected from the group consisting of ‘A’, ‘C’, ‘G’, and ‘T’.

25. The method of claim 17 wherein said bacterial cell is Clostridium thermocellum and said method involves the use of one or more contiguous DNA sequences from the native or modified genome of a Clostridium thermocellum, wherein said one or more contiguous DNA sequences is present in the native or modified genome of Clostridium thermocellum immediately following to the 3′ side of a 5 nucleotide-long continuous sequence of DNA, commonly known to one versed in the art of CRISPR tools as a ‘protospacer adjacent motif’, wherein said 5 nucleotide-long continuous sequence of DNA is selected from the group consisting of 5′-TTTCA-3′, 5′-GGACA-3′, and 5′-AATCA-3′.

26. The method of claim 17 wherein said bacterial cell is Clostridium thermocellum and said method involves the use of one or more contiguous DNA sequences from the native or modified genome of a Clostridium thermocellum , wherein said one or more contiguous DNA sequences is present in the native or modified genome of Clostridium thermocellum immediately following to the 3′ side of a 3 nucleotide-long continuous sequence of DNA, commonly known to one versed in the art of CRISPR tools as a ‘protospacer adjacent motif’, wherein said 3 nucleotide-long continuous sequence of DNA is 5′-NCA-3′, where ‘N’ is a nucleotide selected from the group consisting of ‘A’, ‘C’, ‘G’, and ‘T’.

27. A bacterial cell selected from the group consisting of Clostridium autoethangenum, Clostridium tetani, and Clostridium thermocellum whose native or modified genome was changed by the method of claim 17.

28. A bacterial cell selected from the group consisting of Clostridium autoethangenum, Clostridium tetani, and Clostridium thermocellum whose native or modified genome was changed by the method of claim 18.

29. A bacterial cell selected from the group consisting of Clostridium autoethangenum, Clostridium tetani, and Clostridium thermocellum whose native or modified genome was changed by the method of claim 19.

30. A bacterial cell selected from the group consisting of Clostridium autoethangenum, Clostridium tetani, and Clostridium thermocellum whose native or modified genome was changed by the method of claim 20.

31. A bacterial cell selected from the group consisting of Clostridium autoethangenum, Clostridium tetani, and Clostridium thermocellum whose native or modified genome was changed by the method of claim 21.

32. A bacterial cell selected from the group consisting of Clostridium autoethangenum, Clostridium tetani, and Clostridium thermocellum whose native or modified genome was changed by the method of claim 22.

33. A bacterial cell selected from the group consisting of Clostridium autoethangenum, Clostridium tetani, and Clostridium thermocellum whose native or modified genome was changed by the method of claim 23.

34. A bacterial cell selected from the group consisting of Clostridium autoethangenum, Clostridium tetani, and Clostridium thermocellum whose native or modified genome was changed by the method of claim 24.

35. A bacterial cell selected from the group consisting of Clostridium autoethangenum, Clostridium tetani, and Clostridium thermocellum whose native or modified genome was changed by the method of claim 25.

36. A bacterial cell selected from the group consisting of Clostridium autoethangenum, Clostridium tetani, and Clostridium thermocellum whose native or modified genome was changed by the method of claim 26.

37. A method for identifying protospacer associated motifs of bacteria harbouring endogenous Type I CRISPR genes.

38. A bacterial cell harbouring Type I CRISPR genes whose genome was changed through the use of a protospacer associated motif identified through the use of the method of claim 37.


SEQUENCE LISTING (56 sequences)

SEQ ID NO: 1 - DNA, 32 bp, Clostridium pasteurianum
gtttagccgcggggcagcgcctaaatgtagaa

SEQ ID NO: 2 - DNA, 40 bp, Clostridium pasteurianum
tcagctctcgagcagtcttgaaaagcccctgtattactgc

SEQ ID NO: 3 - DNA, 42 bp, Clostridium pasteurianum
ctactacgatcggtcctaaaagcagggtatgaagtccattag

SEQ ID NO: 4 - DNA, 52 bp, Clostridium pasteurianum
cttgaggtctaggacttctatctgggaatagaatgttgttcgataggcatcc

SEQ ID NO: 5 - DNA, 52 bp, Clostridium pasteurianum
ggatgcctatcgaacaacattctattcccagatagaagtcctagacctcaag

SEQ ID NO: 6 - DNA, 38 bp, Clostridium pasteurianum
gtcaagcgatcggcttagctggtaagaagcaaggtctt

SEQ ID NO: 7 - DNA, 47 bp, Clostridium pasteurianum
gacgatccgcggggttactttttatggataagaaatactcaataggc

SEQ ID NO: 8 - DNA, 30 bp, Clostridium pasteurianum
cctgtagataacaaatacgattcttccgac

SEQ ID NO: 9 - DNA, 40 bp, Clostridium pasteurianum
ggtaaaatttgattgtcctcattgcgatgaagaaagacgt

SEQ ID NO: 10 - DNA, 38 bp, Clostridium pasteurianum
ctttcttcatcgcaatgaggacaatcaaattttaccgc

SEQ ID NO: 11 - DNA, 43 bp, Clostridium pasteurianum
ggttgcaatagaatgtgataaagaccatactcatatgtgacgt

SEQ ID NO: 12 - DNA, 41 bp, Clostridium pasteurianum
cacatatgagtatggtctttatcacattctattgcaaccgc

SEQ ID NO: 13 - DNA, 42 bp, Clostridium pasteurianum
ggataatatggattgaagagtgttcagaagttaaatagacgt

SEQ ID NO: 14 - DNA, 40 bp, Clostridium pasteurianum
ctatttaacttctgaacactcttcaatccatattatccgc

SEQ ID NO: 15 - DNA, 46 bp, Clostridium pasteurianum
ggtttcagtaaaatttgattgtcctcattgcgatgaagaaagacgt

SEQ ID NO: 16 - DNA, 44 bp, Clostridium pasteurianum
ctttcttcatcgcaatgaggacaatcaaattttactgaaaccgc

SEQ ID NO: 17 - DNA, 47 bp, Clostridium pasteurianum
gggtaaaatttgattgtcctcattgcgatgaagaaatagaaagacgt

SEQ ID NO: 18 - DNA, 45 bp, Clostridium pasteurianum
ctttctatttcttcatcgcaatgaggacaatcaaattttacccgc

SEQ ID NO: 19 - DNA, 49 bp, Clostridium pasteurianum
ggaaattgttgcaatagaatgtgataaagaccatactcatatgtgacgt

SEQ ID NO: 20 - DNA, 47 bp, Clostridium pasteurianum
cacatatgagtatggtctttatcacattctattgcaacaatttccgc

SEQ ID NO: 21 - DNA, 49 bp, Clostridium pasteurianum
ggttgcaatagaatgtgataaagaccatactcatatgtttttaagacgt

SEQ ID NO: 22 - DNA, 47 bp, Clostridium pasteurianum
cttaaaaacatatgagtatggtctttatcacattctattgcaaccgc

SEQ ID NO: 23 - DNA, 47 bp, Clostridium pasteurianum
ggtatctataatatggattgaagagtgttcagaagttaaatagacgt

SEQ ID NO: 24 - DNA, 45 bp, Clostridium pasteurianum
ctatttaacttctgaacactcttcaatccatattatagataccgc

SEQ ID NO: 25 - DNA, 47 bp, Clostridium pasteurianum
ggataatatggattgaagagtgttcagaagttaaatatgctggacgt

SEQ ID NO: 26 - DNA, 45 bp, Clostridium pasteurianum
ccagcatatttaacttctgaacactcttcaatccatattatccgc

SEQ ID NO: 27 - DNA, 52 bp, Clostridium pasteurianum
ggtttcagtaaaatttgattgtcctcattgcgatgaagaaatagaaagacgt

SEQ ID NO: 28 - DNA, 50 bp, Clostridium pasteurianum
ctttctatttcttcatcgcaatgaggacaatcaaattttactgaaaccgc

SEQ ID NO: 29 - DNA, 55 bp, Clostridium pasteurianum
ggaaattgttgcaatagaatgtgataaagaccatactcatatgtttttaagacgt

SEQ ID NO: 30 - DNA, 53 bp, Clostridium pasteurianum
cttaaaaacatatgagtatggtctttatcacattctattgcaacaatttccgc

SEQ ID NO: 31 - DNA, 52 bp, Clostridium pasteurianum
ggtatctataatatggattgaagagtgttcagaagttaaatatgctggacgt

SEQ ID NO: 32 - DNA, 50 bp, Clostridium pasteurianum
ccagcatatttaacttctgaacactcttcaatccatattatagataccgc

SEQ ID NO: 33 - DNA, 33 bp, Clostridium pasteurianum
cataacctcagccatatagcttttacctactcc

SEQ ID NO: 34 - DNA, 30 bp, Clostridium pasteurianum
ataggtggattcccttgtcaagattttagc

SEQ ID NO: 35 - DNA, 821 bp, Clostridium pasteurianum
cagtaagcatgacgatccgcggcaaaaaaagcaccgactcggtgccactttttcaagttgataacggactagccttattttaacttgctatttctagctctaaaacatctgtattagcttcatcagatggtggaatgataagggtttgcaccttaatttctcctattgagaaaatcgtctcttctcagacgtcaaaccatgttaatcattgcttttatcaaaaatatatttttaaaattattattaatttattaaatgtcatttttgatacttgttaatgataacacataaaaagtctaaattcaagaacattattactgtctttatgttaaatttattccatacataccataatacaataacaaaattaaatttatatcaattcttccctcatattttttatttaaataagccttaaatcttttcaaattaaagattatttacagaagtcgaggagctactggtaaaatctattgattaaaaaaatatttgtggttataattaaattgtgaattaaataacaatcgatttgtgtatttataagaatataaactttaggaggttactttttatggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacagg

SEQ ID NO: 36 - DNA, 20 bp, Clostridium pasteurianum
ctgatgaagctaatacagat

SEQ ID NO: 37 - DNA, 42 bp, Clostridium pasteurianum
gttttagagctagaaatagcaagttaaaataaggctagtccg

SEQ ID NO: 38 - DNA, 314 bp, Clostridium beijerinckii
ataatctttaatttgaaaagatttaaggcttatttaaataaaaaatatgagggaagaattgatataaatttaattttgttattgtattatggtatgtatggaataaatttaacataaagacagtaataatgttcttgaatttagactttttatgtgttatcattaacaagtatcaaaaatgacatttaataaattaataataattttaaaaatatatttttgataaaagcaatgattaacatggtttgacgtctgagaagagacgattttctcaataggagaaattaaggtgcaaacccttatcattccaccat

SEQ ID NO: 39 - DNA, 109 bp, Clostridium pasteurianum
ggtaaaatctattgattaaaaaaatatttgtggttataattaaattgtgaattaaataacaatcgatttgtgtatttataagaatataaactttaggaggttacttttt

SEQ ID NO: 40 - DNA, 250 bp, Streptococcus pyogenes
atggataagaaatactcaataggcttagatatcggcacaaatagcgtcggatgggcggtgatcactgatgaatataaggttccgtctaaaaagttcaaggttctgggaaatacagaccgccacagtatcaaaaaaaatcttataggggctcttttatttgacagtggagagacagcggaagcgactcgtctcaaacggacagctcgtagaaggtatacacgtcggaagaatcgtatttgttatctacagg

SEQ ID NO: 41 - DNA, 667 bp, Clostridium pasteurianum
cattcagagctcggatggttaacagtgctagaaaatagatatatctataatttaatttagtaaattgataatattcaataagattttaccaagtaagataaaaataaagatacctatgaagtacttatacataaggcttataggtgtttttctattaaaatttacgtaagactaaaaatagctggtaaaatttttgctaaatcctttatttttaatgaatagagcattataattatagtaaagaatggctagttttaagtagttgaaccttaacataggatgtatttaaatcagaaaataagtcttctgtatatactaatcctgattgttgaaccttaacataggatgtatttaaatgttaaaataaaggataaatgattaataaatatgttataatattaattatctaatatttaaattaaggatgcgattttattacggatagaacagagttattaaatgttattaagaatggagaaaattcatatatagaattcaaagaagaagctataaaagcaaaagatttggcagaagaatttgtagcttttgctaatgccgaaggtggaacggtgctaataggaatagctgacgatggaggcataaaaggggtaactgatagtaatatagaagagaagattatgaatatttgcaggaagagctcatcagg

SEQ ID NO: 42 - DNA, 36 bp, Clostridium pasteurianum
cagaaaataagtcttctgtatatactaatcctgatt

SEQ ID NO: 43 - DNA, 30 bp, Clostridium pasteurianum
gttgaaccttaacataggatgtatttaaat

SEQ ID NO: 44 - DNA, 249 bp, Clostridium pasteurianum
ggatggttaacagtgctagaaaatagatatatctataatttaatttagtaaattgataatattcaataagattttaccaagtaagataaaaataaagatacctatgaagtacttatacataaggcttataggtgtttttctattaaaatttacgtaagactaaaaatagctggtaaaatttttgctaaatcctttatttttaatgaatagagcattataattatagtaaagaatggctagttttaagta

SEQ ID NO: 45 - DNA, 30 bp, Clostridium pasteurianum
atttaaatacatcctatgttaaggttcaac

SEQ ID NO: 46 - DNA, 30 bp, Clostridium autoethanogenum
atttaaatacatctcatgttgaggttcaac

SEQ ID NO: 47 - DNA, 30 bp, Clostridium autoethanogenum
atttaaatacatcttatgttgaggttcaac

SEQ ID NO: 48 - DNA, 31 bp, Clostridium tetani
aatttacattccaatatggtgctactaatac

SEQ ID NO: 49 - DNA, 30 bp, Clostridium tetani; identical to SEQ ID NO: 45

SEQ ID NO: 50 - DNA, 30 bp, Clostridium tetani
atttaaatacaactcttgttattgttcaac

SEQ ID NO: 51 - DNA, 30 bp, Clostridium thermocellum
gtttgtatcgtacctatgaggaattgaaac

SEQ ID NO: 52 - DNA, 30 bp, Clostridium thermocellum
gtttttatcgtacctatgaggaattgaaac

SEQ ID NO: 53 - DNA, 30 bp, Clostridium thermocellum
gtttcaattcctcataggtacgataaaaac

SEQ ID NO: 54 - DNA, 37 bp, Clostridium thermocellum
gttgaagtggtacttccagtaaaacaaggattgaaac

SEQ ID NO: 55 - DNA, 37 bp, Clostridium thermocellum
gttgaagaggtacttccagtaaaacaaggattgaaac

SEQ ID NO: 56 - DNA, 298 bp, Clostridium pasteurianum
gttaaaataaaggataaatgattaataaatatgttataatattaattatctaatatttaaattaaggatgcgattttattacggatagaacagagttattaaatgttattaagaatggagaaaattcatatatagaattcaaagaagaagctataaaagcaaaagatttggcagaagaatttgtagcttttgctaatgccgaaggtggaacggtgctaataggaatagctgacgatggaggcataaaaggggtaactgatagtaatatagaagagaagattatgaatatttgcaggaa